SEQUENCE LISTING (6 sequences)

SEQ ID NO: 1 - protein, 10 aa, synthetic construct
GGGQHREDGS

SEQ ID NO: 2 - protein, 7 aa, synthetic construct
QHREDGS

SEQ ID NO: 3 - protein, 36 aa, synthetic construct
LGDFFRKSKEKIGKEFKRIVQRIKDFLRNLVPRTES

SEQ ID NO: 4 - protein, 17 aa, synthetic construct
GFKRIVQRIKDFLRNLV

SEQ ID NO: 5 - protein, 12 aa, synthetic construct
KRIVQRIKDFLR

SEQ ID NO: 6 - protein, 19 aa, synthetic construct
EEQIGKSSTRGRKCCRRKK

(19) United States
(12) Patent Application Publication
(10) Pub. No.: US 2026/0027192 A1
(43) Pub. Date: Jan. 29, 2026

(54) COMBINED DELIVERY OF ANTIGENS AND TOLEROGENIC SIGNALS VIA DUAL-SIZED HYDROGEL SPHERES AND MOF COMPOSITES FOR TYPE-1 DIABETES VACCINE DEVELOPMENT

(71) Applicant: THE HONG KONG UNIVERSITY OF SCIENCE AND TECHNOLOGY, Hong Kong (CN)

(72) Inventors: Jin Teng CHUNG, Hong Kong (CN); Chi Ming Laurence LAU, Hong Kong (CN); Ying CHAU, Hong Kong (CN)

(21) Appl. No.: 19/143,578

(22) PCT Filed: Dec. 28, 2023
(86) PCT No.: PCT/IB2023/000798

§ 371 Date: Jun. 26, 2025

Related U.S. Application Data
(60) Provisional application No. 63/477,559, filed on Dec. 28, 2022.

(51) Int. Cl. A61K 39/00; A61K 9/00; A61K 9/06; A61K 38/20; A61K 38/28; A61K 47/69; A61P 37/06

(52) U.S. Cl. CPC A61K 39/0008; A61K 9/0019; A61K 9/06; A61K 38/206; A61K 38/28; A61K 47/6939; A61P 37/06; A61K 2039/54; A61K 2039/55555; A61K 2039/575; A61K 2039/577

(57) ABSTRACT

It pertains to a novel platform for the sustained release of Type-1 diabetes antigens. More specifically. it provides a combination of dual sized hydrogel particles for the delivery of Type-1 diabetes antigens and multi-component adjuvants to confer immune tolerance.

### CROSS-REFERENCE TO RELATED APPLICATION

This application claims the benefit of U.S. Provisional Application Ser. No. 63/477,559, filed Dec. 28, 2022, which is hereby incorporated by reference in its entirety including any tables, figures, or drawings.

### REFERENCE TO SEQUENCE LISTING

The Sequence Listing for this application is labeled “HKUS184XPCT.xml” which was created on Dec. 27, 2023 and is 6,257 bytes. The entire content of the Sequence Listing is incorporated herein by reference in its entirety.

### BACKGROUND OF THE INVENTION

Type-1 diabetes (TID) is an autoimmune disorder, affecting almost 4.9-million people worldwide in 2020. In 2015, the prevalence of diabetes in Hong Kong was 8.4%, of which 0.5% was TID. TID is an autoimmune disease, characterized by the destruction of insulin-secreting β-cells in the pancreas by immune cells. The reduction in plasma insulin level results in hyperglycemia and diabetic complications. Patients rely on long-term insulin replacement as standard of care to alleviate diabetic symptoms; nonetheless, the cellular and humoral responses against β-cells and insulin persist. Clinical studies have turned to immunotherapy to reverse TID. The existing approaches include the use of broad spectrum immunosuppressants, broad T-cell suppression with anti-CD3, CD4 and CD20 antibody administrations, CAR-T regulatory cells development, and, more recently, the TID vaccines [1].

Several autoantibodies have been identified that act against insulin, including GAD65, IA-2, and ZnT8 [2]. Studies have utilized these antigens for the development of TID vaccines [3]. The principle of the use of vaccines to treat T1D comprises presenting the antigens in a non-inflammatory context to induce tolerance, taming the immune flares against β-cells and insulin [4]. TID therapeutic vaccine outcomes are expected to cause tolerogenic dendritic cell (DC) induction, tolerogenic cytokine profile (IL-10, PD-1 and CTLA-4), reduction of Th1 activity, increase in Th2 and T-regulatory cell activity, reduction of autoantibodies, blockade of β-cells and effector T-cell interactions targeting β-cells, and immune memory for long-term protection [3], [5]. GAD65 and insulin are the two major autoantigens in T1D. The administration of a GAD65 isoform could suppress immune destruction of β-cells in non-obese diabetic (NOD) mouse model. Moreover, the co-administration of GAD65 with adjuvants such as alum (in Diamyd®), Kynurenine (IDO analogue), cholera toxin B-subunit and IL-10, GM-CSF and CpG could suppress T1D progression [6]-[9]. In addition, the use of insulin can act as a vaccine in the context of tolerogenic adjuvants, such as GM-CSF, TGF-β, IL-10, rapamycin, and vitamin D3, were also effective in treating T1D [10]-[12] [13]. However, there remains a need for a platform for the sustained release of T1D antigens to induce tolerance of autoantibodies.

### BRIEF SUMMARY OF THE INVENTION

The subject invention relates to a platform for the sustained release of T1D antigens to a subject. In certain embodiments, the platform can be used to induce tolerance differentiation of dendritic cells (DCs). DCs are important target in vaccine development because they uptake, process, and present antigens on MHC-I and II to CD8⁺ and CD4⁺ T-cells, respectively, regulating the adaptive immunity. More importantly, the maturation status of DCs determines the immune outcome. DCs mature in the presence of anti-inflammatory signals to develop a tolerogenic phenotype, which induces T-regulatory responses and tolerance [14].

In certain embodiments, the platform comprises a mixture of hydrogel microparticles (HMPs) that create an anti-inflammatory niche for the tolerogenic maturation of resident DCs and hydrogel nanoparticles (HNPs) that improve antigen uptake and trafficking within the cells. In certain embodiments, the HMP can comprises a tolerogenic adjuvant, such as, for example, IL-10. In certain embodiments, IL-10 has demonstrated a broad immunosuppressive effect on immune cells, promotes tolerogenic DC generation, and the resulting DC-10 cells generate more IL-10 endogenously [15], [16]. In certain embodiments, the tolerogenic adjuvant can be loaded in a zwitterionic motif on the surface of the HMP. In certain embodiments, the tolerogenic adjuvant in the zwitterionic motif can suppress the rapid burst release of the tolerogenic adjuvant from the hydrogel polymer network.

In certain embodiments, the subject hydrogel particle system delivers antigens and modulates the innate immune responses in a subject, such as, for example, at the subcutaneous injection site of the hydrogel particle system. In certain embodiments, the antigen can be loaded with the HNPs to coat the HNP, such as, for example, with Q-peptide, which is derived from an anti-inflammatory protein Angpt-1. In certain embodiments, the HNPs can be internalized by resident antigen present cells (APCs), serving as intracellular antigen depots to maintain MHC-presentation of antigens and immunomodulation.

In certain embodiments, the subject hydrogel particle system can suppress the immunogenic maturation of DCs and direct DC trafficking antigens to inguinal lymph nodes (ILN) and the spleen to induce a greater level of T-regulatory cells than other groups that have not been treated with the subject hydrogel particle system. As a result, in certain embodiments, the antigen-specific humoral and cellular responses were greatly inhibited.

In certain embodiments, the tolerogenic adjuvant in the zwitterionic motif can induce anti-inflammatory responses on resident APCs, inducing a greater production of T-regulatory cells, thereby extending the tolerogenic response against an antigen of interest, including the antigen on the surface of the HNP.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1G. Physical characterizations of HNPs. FIGS. 1A-1B: The mean diameter and surface charge of NP and tL-NP were measured at pH 5, 7 and 10 with NTA Zeta View. Particles were incubated in ddH2O for 10 mins at room temperature, with pH adjustment by 1mM NaOH and HCl. FIG. 1C: The structure of modified Q-peptide (GGGQHREDGS; SEQ ID NO: 1) and predicted isoelectric point (pI) and hydrophilicity. FIG. 1D: The size distribution of particles post-incubation in ddH2O of various pH. FIGS. 1E-1F: The cumulative release % of total encapsulated F-insulin (6 kDa) and F-OVA (45 kDa) from NPs incubated in PB buffer of various pH. (N=3, graphs are presented as mean #s.d.) FIG. 1G: The schematic illustration of the swelling and release of antigens, F-insulin, and F-OVA from HNPs with increasing H concentration and time.

FIGS. 2A-2C. Physical characterizations of HMPs. FIG. 2A: The size and distribution of HMPs, zDX and zDX/MOF. F-OVA was loaded in particles, and they were imaged under fluorescence microscope (20× magnification, FITC channel). The microscope image was analysed with ImageJ software to estimate the particles size and number. Scale bar represents 100 μm. Schematic illustration of HMPs prepared with water-in-oil bulk emulsion method and strained with cell strainer to isolate HMPs in the size range of 50 to 100 μm for cell culture and animal treatments. FIG. 2B: The cumulative release % of F-lysozyme (F-Lyz) from zDX and zDX/MOF incubated in PBS, pH 7.4, 37° C. FIG. 2C: The average cumulative release % of total encapsulated IL-10 from zDX/MOF incubated in 0.1M PB buffer of various pH, containing 1% BSA at 37° C. The amount of IL-10 released was measured with ELISA. (N=2).

FIGS. 3A-3E. The antigen uptake and presentation by JAWSII. FIG. 3A: The viability of JAWSII at 96-hour post-treatment with various vaccine formulations. Cells were stained with propidium iodide and analyzed with flow cytometry. FIGS. 3B-3C: The antigen uptake profile of cells was probed with F-OVA, loaded in tL-NP or NP, with or without co-delivery of IL-10 laden zDX/MOF HMPs. For negative control, cells were treated with bolus OVA with or without soluble IL-10 for 24 hours. Cells were replaced with fresh medium. The treatment schedule is summarized in Table 2 and FIG. 10. The F-OVA⁺ cell population % and mean fluorescence intensity (MFI) of F-OVA in total gated viable cells are shown. (N>4, graphs are presented as mean±s.d., * is p<0.05 in comparison to bolus OVA at respective timepoint). FIG. 3D: The live cell confocal microscopy of JAWSII treated with OVA-laden NP, tL-NP, and bolus OVA. F-OVA (green) was used to probe intracellular antigen distribution and lysosomes (red) were visualized with Lysotracker-Red. Cells were imaged at 24 and 96-hour post-treatment and cells were washed with 0.001% Triton-X100 at 24-hour, after imaging to remove un-internalized antigens. Scale bar represents 10 μm. FIG. 3E: The overlapping coefficient of green and red channels (yellow) to measure the co-localization of F-OVA in lysosomes. At 96-hour, no signal was detected in cells treated with bolus OVA. (N=4, mean±s.d., +is p<0.05 in comparison to 96-hour treatment of relevant groups).

FIGS. 4A-4H. JAWSII maturation profile. FIGS. 4A-4E: The expression profile of surface markers, MHC-II, CD40, CD86, CD206 and CCR7 were measured with flow cytometry. Cells were treated with various vaccine formulations for 96 hours. For negative control, cells were treated with bolus OVA with or without soluble IL-10 for 24 hours. Cells were then replaced with fresh medium. The treatment schedule is summarized in Table 2. All graphs are presented as mean fluorescence intensity (MFI) normalized to untreated cells±s.d. (N=4, * is p<0.05 in comparison to bolus OVA at respective timepoints). FIGS. 4F-4H: The average production level of cytokines, IL-6, TNF-α and IL-10 was measured with ELISA at various timepoints post-treatment. (N=2).

FIGS. 5A-5H. Biodistribution of OVA post s.c. injection. FIGS. 5A-5F: C57BL\6 mice were injected s.c. with same dosage of IR-labelled OVA in various forms, tL-NP, NP, alum, or bolus, prepared in sterile PBS. Mice in sham group were injected with PBS and the organs were used as blank to define the signal threshold in IR-scanning. Mice were sacrificed on day 7, 14 and 21 post-immunization and the organs were isolated for ex vivo IR-imaging to monitor the antigen distributions. Representative heat maps of various organs, skin at injection site, ILN, spleen, liver, kidneys, and lungs were shown. The scale bar of signal intensity was shown. FIGS. 5G-5H: The population percentage of CD11c⁺ MHC-II⁺, indicating activated DCs in ILN and spleen were analyzed with flow cytometry. Mice were sacrificed on day 7, 14, and 21 post-immunization and the organs were isolated and processed to obtain single cell populations for flow cytometry. (N=4, * is p<0.05 in comparison to bolus OVA at respective timepoints).

FIGS. 6A-6D. Cellular and humoral response in animals, post-OVA immunization. C57BL\6 mice were injected s.c. with various OVA vaccine formulations, such as OVA-laden tL-NP, NP, alum, and bolus OVA. OVA was maintained at same dosage at 500 μg per animal (average weight of 22 g). Mice in sham group were injected with PBS. FIGS. 6A-6B: T-cell population distribution in ILN and spleen on day 7 post-immunization. Graphs are presented as percentage of gated populations of CD3⁺CD4⁺, CD3⁺CD8⁺ and CD4⁺CD25⁺ post-immunization with various OVA formulations. (N>4, where * is p<0.05 in comparison to bolus OVA). FIGS. 6C-6D: The antibody titer of serum anti-OVA IgGI and IgG2a on day 7, 14, and 28 post-immunizations with various OVA vaccine formulations. (N=2).

FIG. 7. MALDI-TOF and chemical structure of GGGQHREDGS (SEQ ID NO: 1).

FIG. 8. Fluorescent images of protein/MOF composites. 0.25 mg/mL fluorescently labelled proteins, OVA, lysozyme (Lyz) and insulin in PBS, prepared in 0.32M 2-methyl imidazole and 0.02M zinc acetate. The ZIF-8 mineralized proteins were incubated at room 30 temperature overnight, washed in PBS. The protein/MOF composites were resuspended in 2% wt/V zDX solution and imaged under fluorescent confocal microscopy at 63X. The scale bar represents 50 μm.

FIG. 9. Graphical representation of the 1) the extracellular HMPs for the loading of a small cytokine in zwitterionic DX-based hydrogel)/MOF composites to suppress the rapid burst release of IL-10 from the loose hydrogel polymer networks, and 2) intracellular HNPs for the continuous release of antigens for MHC processing and presentation.

FIG. 10. The treatment schedule of dual sized sphere based T1D vaccines on JAWSII in vitro.

### BRIEF DESCRIPTION OF THE SEQUENCES

SEQ ID NO: 1: Q-peptide derivative

SEQ ID NO: 2: Angiopoietin-1-derived peptide Q-peptide

SEQ ID NO: 3: LL-37 peptide

SEQ ID NO: 4: GF-17 peptide

SEQ ID NO: 5: KR-12 peptide

SEQ ID NO: 6: defensin peptide

### DETAILED DISCLOSURE OF THE INVENTION

The subject invention relates to a platform for the sustained release of T1D antigens in to induce tolerance differentiation of dendritic cells.

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

In this application, the terms “polypeptide”, “peptide”, and “protein” are used interchangeably herein to refer to a polymer of amino acids. The terms apply to amino acid polymers in which one or more amino acid residues are artificial chemical mimetics of corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, the term “amino acid” refers to standard nomenclature, amino acid residue as denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, “adjuvants” refer to any substance that can enhance the subject's immune response to an antigen.

As used herein, “antigen” refers to any toxin or other foreign substance that can induce an immune response in the subject.

As used herein, the term “hydrogel” refers to a substance formed when an organic polymer (natural or synthetic) is cross-linked via covalent, ionic, or hydrogen bonds to create a three-dimensional open-lattice structure which entraps water molecules to form a gel.

“Subject” refers to an animal, such as a mammal, for example a human. The methods described herein can be useful in both humans and non-human animals. In some embodiments, the subject is a mammal (such as an animal model of disease), and in some embodiments, the subject is a human. The terms “subject” and “patient” can be used interchangeably. The animal may be for example, humans, pigs, horses, goats, cats, mice, rats, dogs, apes, fish, chimpanzees, orangutans, guinea pigs, hamsters, cows, sheep, birds, chickens, as well as any other vertebrates.

“Treatment”, “treating”, “palliating” and “ameliorating” (and grammatical variants of these terms), as used herein, are used interchangeably. These terms refer to an approach for obtaining beneficial or desired results including but not limited to therapeutic benefit. A therapeutic benefit is achieved with the eradication or amelioration of one or more of the physiological symptoms associated with the underlying disease such that an improvement is observed in the patient, notwithstanding that the patient may still be afflicted with the disease.

As used herein, “Type-1 diabetes” and “T1D” are used interchangeably to refer to the chronic condition wherein the pancreas produces little or no insulin.

Any compositions or methods provided herein can be combined with one or more of any of the other compositions and methods provided herein.

Other features and advantages of the invention will be apparent from the following description of the preferred embodiments thereof, and from the claims.

All references cited herein are hereby incorporated by reference in their entirety.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### Biopolymer Compositions

The subject hydrogels are formed from polymers that provide chemically defined and versatile support for peptides or proteins. The properties of the hydrogels can be changed by varying polymer structures, polymer molecular weight, and cross-linking reaction conditions. In certain embodiments, the components of the hydrogel can be provided either separately or combined, in dry form, such as powders, or in solution form. The hydrogel forms when cross linking conditions are applied. For example, the precursor components of the hydrogel are dissolved in a liquid, such as water, and the solution temperature is increased to induce cross linking and form the hydrogel.

In certain embodiments, the subject bead-based, hydrogel platform can comprise a polymer, such as, for example, a hydrophilic polymer or a polysaccharide, such as, for example, cyclodextrin, alginate, dextran, hyaluronic acid, pullulan, PEG or any combination thereof. In preferred embodiments, the hydrogel comprises dextran. In certain embodiments, each hydroxyl group can be chemically modified or at least 1, 2, 3, 4 5, 6, 7, 8, 9, 10, or more hydroxyl groups of the polysaccharide can by chemically modified. In certain embodiments, the polysaccharide is modified with various functional groups, such as, for example, alkyl halide, carboxylate, alcohol, epoxide, or vinyl sulfone groups (-VS), and, optionally, subsequently with DTT to create, for example, free thiol groups (-SH); or modified with carbazate. In certain embodiments, the polysaccharide is oxidized into aldehydes. In certain embodiments, the degree of modification can be measured by, for example, 1H-NMR spectroscopy and/or colorimetric methods, such as, for example Ellman's assay. In certain embodiments, the degree of modification can be about 1% to about 50%. In certain embodiments, the degree of modification can be altered by changing the reaction time and/or the degree of oxidation by changing the amount of the oxidant, such as, for example, NaIO₄. In certain embodiments, each about 1 g of the polysaccharide or hydrophilic polymer can be reacted with about 0.3 mmol to about 6.2 mmol of the oxidant. In preferred embodiments, the HNP comprises dextrose crosslinked via carbazate-aldehyde condensations to form an acid-sensitive hydrazone bond.

In certain embodiments, the hydrogel comprises hydrogel nanoparticles (HNPs), with a size range of about 100 nm to about 300 nm in diameter, and hydrogel microparticles (HMPs) have a size range of about 10 μm to about 100 μm or about 50 μm. In certain embodiments, the HNP to HMP ratio is about 1×10³:1 to about 1×10⁶:1. In certain embodiments, the HNPs can be formed using mini-inversed (water-in-oil) emulsion under ultrasonication, membrane extrusion, microfluidics droplet-synthesis, lithography, electrodynamic spraying, or mechanical fragmentation. In certain embodiments, protein solutions containing antigens and cytokines were prepared in a buffer, such as, for example, PBS, HEPES, or Tris, at a pH of about 4 to about 8 with a modified polymer, such as, for example, a carbazate-modified polysaccharide, emulsified in an oil phase (i.e., continuous phase) consisting of 75% to about 98%, about 80% to about 98%, about 85% to about 98%, about 90% to about 98%, or about 95% to about 98% v/v n-heptane, mineral oil, cyclohexane, octanol, or hexane and a surfactant, such as, for example, Span 80, Tween 80, saponin, Triton-X, sorbitan, or any combination thereof. In certain embodiments, two surfactants can be used at a ratio of about 250:1, about 200:1, about 150:1, about 100:1, about 50:1, about 25:1, about 10:1, about 3:1, about 1:1. In preferred embodiments, the surfactant of the continuous phase comprises about 1.5% Span 80 and 0.5% Tween 80. In certain embodiments, an oxidized polymer is prepared separately in a buffer, such as, for example, PBS, emulsified in the same conditions as the carbazate-modified polysaccharide, and added dropwise into the modified polymer/protein solution mixture and sonicated for about 2 min on ice (e.g., at about 0° C.). In certain embodiments, the mixture of the modified polymer/protein solution and oxidized polymer can be stirred at room temperature (e.g., about 18° C. to about 25° C.) for about 6 hours to about 24 hours and the washed to remove surfactants and organic solvents, in, for example, a solution comprising in n-heptane, isopropanol, ethanol, acetone, and methanol. In certain embodiments, ultracentrifugation at about 200,000 r.c.f. for about 15 min can be applied to remove the supernatant.

In certain embodiments, at least 1, 2, 3, 4, 5, 6, 7, 8, 9, 10, or more peptides can be bound to the surface of the HNP. In certain embodiments, the peptide is an Angiopoietin-1-derived peptide, such as, for example, QHREDGS (SEQ ID NO: 2) or GGGQHREDGS (SEQ ID NO: 1), insulin, GAD65, LL-37 (LGDFFRKSKEKIGKEFKRIVQRIKDFLRNL VPRTES; SEQ ID NO: 3), cathelicidins (e.g., GF-17: GFKRIVQRIKDFLRNLV (SEQ ID NO: 4) or KR-12: KRIVQRIKDFLR (SEQ ID NO: 5)), defensins (e.g., EEQIGKSSTRGRKCCRRKK; SEQ ID NO: 6), or ovalbumin. In certain embodiments, the peptide can be bound on the HNP at the N-terminus of the peptide to consume an excess aldehyde groups of the HNP to form an imine bond. In certain embodiments, the resulting HNPs can be washed in methanol to remove unreacted peptides. In certain embodiments, the resulting HNP has a mean diameter of about 100.0 nm to about 400.0 nm or about 150.0 nm to about 300.0 nm. In certain embodiments, the resulting HNP is cationic or anionic.

In certain embodiments, the HMP can be formed in water-in-oil bulk emulsions, which does not involve ultrasound that can further disperse the droplets into nano-sized range. In certain embodiments, to form the HMP, polymers, such as, for example, dextran, can be crosslinked with DTT or another small molecule that can crosslink the hydrogel precursors, including, for example, ethylene-dithiol, dithiol alternatives, ethylene-diamine and diamine alternatives, which is pre-dissolved in oil phase, via thiol-Michael addition. In certain embodiments, a metal-organic framework (MOF) can be bound to the surface of the HMP. In certain embodiments, the MOF is a Zeolitic imidazolate framework (ZIF) CuTCPP, NU-1000, MIL-100, NPCN-333, or PCN-222/MOF-545. IN certain embodiments, the ZIF is ZIF-8, ZIF-90, or amorphous ZIF. In certain embodiments, the MOF can comprise a protein, such as, for example, lysozyme, TGF-β, IL-15, IL-21, IL-4, IL-11, IL-13, or IL-10.

In certain embodiments, the HMPs have a diameter of about 50 μm to about 100 μm, preferably, about 70 μm to about 90 μm in diameter.

In certain embodiments, the subject platform can be formed by mixing the HNP and the HMP. In certain embodiments, HNP and the HMP can be mixed at a ratio of about 1:1 and 1:10, about 1:1 and about 1:9, about 1:1 and about 1:8, about 1:1 and about 1:7, about 1:1 and about 1:6, about 1:1 and about 1:5, about 1:1 and about 1:4, about 1:1 and about 1:3, about 1:1 and about 1:2, about 1:1 and about 1:1.5. In certain embodiments, HMP and the HNP can be mixed at a ratio of about 1:1 and 1:10, about 1:1 and about 1:9, about 1:1 and about 1:8, about 1:1 and about 1:7, about 1:1 and about 1:6, about 1:1 and about 1:5, about 1:1 and about 1:4, about 1:1 and about 1:3, about 1:1 and about 1:2, about 1:1 and about 1:1.5. In certain embodiments, the HNP to HMP ratio is about 1×10³:1 to about 1×10⁶:1.

In certain embodiments, the polymer can have a weight per volume concentration in the hydrogel microparticle of about 5.0% to about 50%, about 7.5% to about 30%, or about 15%. In certain embodiments, the polymer can have a weight per volume concentration in the hydrogel nanoparticle of about 5.0% to about 50%, about 7.5% to about 30%, or about 15%.

### Methods of Use

The subject invention relates to a novel, particle platform that can be used to deliver various immune active peptides to a subject. In certain embodiments, the platform comprises hydrogel microparticles (HMPs) and hydrogel nanoparticles (HNPs).

In certain embodiments, the HMP and HNP can be co-cultured with an innate immune cell, such as, for example, an immature dendritic cell line (e.g., JAWSII) or a macrophage cell line (e.g., RAW Blue™, InvivoGen, San Diego, CA), to investigate the uptake and intracellular distributions of the peptides of the HNPs and the HMPs. In certain embodiments, the interaction between the peptides and the immune cell can be visualized using a probe conjugated to the peptide, such as, for example, a fluorescein (FITC), rhodamine, cyanine, BODIPY, or naphthalimide probe. In certain embodiments, lysosomes and late endosomal compartments can be visualized.

In certain embodiments, the subject platform can be used as a vaccine delivery vehicle and depot for the prolonged release of antigens and subunits in a subject. In certain embodiments, the subject hydrogel can be administered via subcutaneous, intramuscular, or intratumoral route. In certain embodiments, the subject hydrogel containing an antigen can be administered at least 1, 2, 3, 4, 5, 6, 7, 8, 9, 10, or more times. In certain embodiments, the time period between doses of the subject hydrogel containing an antigen can be at least about 3 hours, about 6 hours, about 12 hours, about 1 day, about 2 days, about 3 days, about 1 week, about 2 weeks, about 3 weeks, about 4 weeks, about 6 weeks, about 2 months, about 3 months, about 6 months, about 9 months, about 1 year, about 18 months, about 2 years, about 3 years, about 5 years, or about 10 years. In certain embodiments, humoral immunity, such as, for example, antigen specific IgGI and IgG2 levels in blood plasma, can be measured and systemic cytokine levels, such as, for example, serum IL-6, IL-1β, IFN-γ, TNF-α, IL-10, BAFF, or any combination thereof can be monitored post-vaccination. In certain embodiments, cellular immunity, such as, for example, T-cell population and subtype profiling in secondary lymphoid organs, such as, for example, lymph node and spleen can be monitored post-vaccination. In certain embodiments, cytolytic activity of CD8-T and NK cells can also be monitored post-vaccination.

In some embodiments, a first dose is administered at the same concentration as at least one second dose. In some embodiments a first dose is administered at a different concentration from at least one second dose. In preferred embodiments, a first dose is administered at a lower concentration than at least one second dose.

The skilled artisan will understand that the dosage of the compositions of the instant invention varies, depending upon, for example, the route of administration, the particular platform comprising HNPs and HMPs to be used in the composition, other drugs being administered, and the age, condition, gender and seriousness of the disease in the subject as described above. An effective dose of a platform comprising HNPs and HMPs of the invention generally ranges between about 1 μg/kg of body weight and 100 mg/kg of body weight. Examples of such dosage ranges include, but are not limited to, about 1.5 μg/kg to about 90 mg/kg; about 2 μg/kg to about 80 mg/kg; about 5 μg/kg to about 70 mg/kg; about 7.5 μg/kg to about 65 mg/kg; about 10 μg/kg to about 60 mg/kg; about 12.5 μg/kg to about 55 mg/kg; about 15 μg/kg to about 50 mg/kg; about 17.5 μg/kg to about 45 mg/kg; about 20 μg/kg to about 40 mg/kg; about 22.5 μg/kg to about 35 mg/kg; about 25 μg/kg to about 30 mg/kg; about 27.5 μg/kg to about 25 mg/kg; about 30 μg/kg to about 20 mg/kg; about 32.5 μg/kg to about 18 mg/kg; about 35 μg/kg to about 17 mg/kg; about 37.5 μg/kg to about 16 mg/kg; about 40 μg/kg to about 15 mg/kg; about 42.5 μg/kg to about 14 mg/kg; about 45 μg/kg to about 13 mg/kg; about 47.5 μg/kg to about 12 mg/kg; about 50 μg/kg to about 11 mg/kg; about 52.5 μg/kg to about 10 mg/kg; about 55 μg/kg to about 9 mg/kg; about 57.5 μg/kg to about 8 mg/kg; about 60 μg/kg to about 7 mg/kg; about 62.5 μg/kg to about 6 mg/kg; about 65 μg/kg to about 5 mg/kg; about 67.5 μg/kg to about 4 mg/kg; about 70 μg/kg to about 3 mg/kg; about 72.5 μg/kg to about 2 mg/kg; about 75 μg/kg to about 1 mg/kg; about 77.5 μg/kg to about 800 μg/kg; about 80 μg/kg to about 700 μg/kg; about 82.5 μg/kg to about 600 μg/kg; about 85 μg/kg to about 500 μg/kg; about 87.5 μg/kg to about 400 μg/kg; about 90 μg/kg to about 300 μg/kg; about 92.5 μg/kg to about 200 μg/kg; about 95 μg/kg to about 100 g/kg.

In certain embodiments, the platform comprising HNPs and HMPs of the invention are administered at a dose of about 50 μg/kg to about 200 μg/kg, preferably, about 75 μg/kg to about 150 μg/kg. or, most preferably, about 100 μg/kg to about 120 μg/kg.

In some embodiments, the therapeutically effective amount of a platform comprising HNPs and HMPs of the invention can be administered through subcutaneous, transdermal, intramuscular, or intratumoral administration.

In certain embodiments, the platform compositions of the instant invention may be formulated for parenteral administration e.g., by injection, for example, bolus injection or continuous infusion. In addition, the compositions may be presented in unit dose form in ampoules, pre-filled syringes, and small volume infusion or in multi-dose containers with or without an added preservative. The compositions may be in forms of suspensions, solutions, or emulsions in oily or aqueous vehicles. The composition may further contain formulation agents such as suspending, stabilizing and/or dispersing agents. In further embodiments, the active ingredients of the compositions according to the instant invention may be in powder form, obtained by aseptic isolation of sterile solid or by lyophilization from solution for constitution with a suitable vehicle, e.g. sterile, pyrogen-free water, before use.

In certain embodiments, the HMPs with zwitterionic phosphorylcholine moieties (PC) can circumvent responses mediated by macrophages and fibroblasts in a subject, which results in the fibrotic encapsulation of HMPs, isolating, for example, the IL-10 secreting device from the host tissue environment. In certain embodiments, the HNPs with anti- inflammatory peptide, Gln-His-Arg-Glut-Asp-Gly-Ser-NH₂ (QHREDGS, or Q-peptide; SEQ ID NO: 2). Q-peptide is derived from angiopoietin-1 (Angpt-1), a ligand for β1 and β3 integrins, promotes vascular integrity and remodelling to maintain the barrier functions of blood vessels during injury and inflammation.

In certain embodiments, the antigen on the surface of the HNP can be released after administration to a subject over a time period of at least about 1 hour, about 2 hours, about 3 hours, about 4 hours, about 5 hours, about 6 hours, about 7 hours, about 8 hours, about 9 hours, about 10 hours, about 11 hours, about 12 hours, 24 hours, 48 hours, 72 hours, or 96 hours.

In certain embodiments, the peptide encapsulated with the MOF on the surface of the HMP can be released after administration to a subject over a time period of at least about 6 hours, about 12 hours, about 18 hours, about 1 day, about 2 days, about 3 days, about 4 days, about 5 days, about 6 days, about 7 days, about 8 days, about 9 days, about 10 days, about 11 days, about 12 days, 14 days, 18 days, 21 days, 28 days, 29 days, 30 days, or 31 days.

In certain embodiments, the administration of the HNPs and HMPs to the subject can differentiate dendritic cells into immunogenic or tolerogenic phenotypes. In certain embodiments, expression of CD86, CD40, MHC-II, IL-6, CD206, CCR7, and TNF-α can be measured in the subject before and after administration of the HNPs and HMPs. In certain embodiments, other tolerogenic markers include a reduction of IL-12, IL-1b and/or other pro-inflammatory cytokines level and/or an increase in IL-10, TGF-b, IL-1RA and/or other anti-inflammatory markers. In certain embodiments, levels of CD4⁺CD25⁺ cells, CD3⁺CD4⁺ cells and CD3⁺CD8⁺ cells can be measured in the subject before and after administration of the HNPs and HMPs. In certain embodiments, the cellular immunity is characterized by an increased level of CD4+FOXP3+ T-cells and/or other T-cell markers, including CTLA-4, Lag3, secretion of IL-10, TGF-β, and/or IL-35. In certain embodiments, levels of IgG1 and IgG2a can be measured in the subject before and after administration of the HNPs and HMPs. In certain embodiments, humoral immunity can also be characterized by the phenotype and functionality of regulatory β-cells (Bregs) subtype producing IL-10, TGF-b, IL-35, and granzymes. In certain embodiments, tolerogenic DCs can characterized by low expression level of co-stimulatory molecules, CD86 and CD40, and production of anti-inflammatory cytokine IL-10.

### MATERIALS AND METHODS

### Hydrogel Nanoparticles

The subject dual hydrogel particle system consisted of nanoparticles (NPs) and microparticles (MPs). NPs were designed to be in the range of 100 to 300 nm in diameter for phagocytosis or receptor-mediated endocytosis by resident innate immune cells, such as dendritic cells, macrophages, and monocytes. The antigen (insulin and GAD65 or model antigen-ovalbumin)-laden NPs were aimed for targeting and participating in the intracellular trafficking, processing, and presentation of antigens. Conversely, the MPs were constructed to have mean diameter of >50 μm to suppress cell uptake and decorated with zwitterionic motifs to inhibit foreign body responses which lead to fibrotic encapsulation of hydrogel system. For controlled release of IL-10, an immunosuppressive cytokine, ZIF-8, was incorporated in MPs to suppress the rapid burst release of small cytokines.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### NP Physical Characterizations

We successfully synthesized DX-based NPs. DX was oxidized with NaIO₄ to form aldehydes or modified with carbazate. The polymers crosslinked to form hydrogel network via the condensation of aldehyde and carbazate. The resulting hydrazone bonds were sensitive to acid degradation. NPs were formed in mini-inversed (water-in-oil) emulsion under ultrasonication. Briefly, protein solutions were prepared in PBS, pH 4 with carbazate-modified DX, emulsified in oil phase consisting of 98% n-heptane, 1.5% Span 80 and 0.5% Tween 80. Oxidized DX was prepared separately in PBS, emulsified, and added dropwise into the DX-carbazate/protein mixture (F-OVA, F-insulin), and continue to sonicate for 2 min on ice. The mixture was stirred in room temperature overnight, washed extensively in n-heptane, isopropanol, and methanol to remove surfactants and organic solvents. Ultracentrifugation at 200,000 r.c.f. for 15 min was applied to remove supernatant.

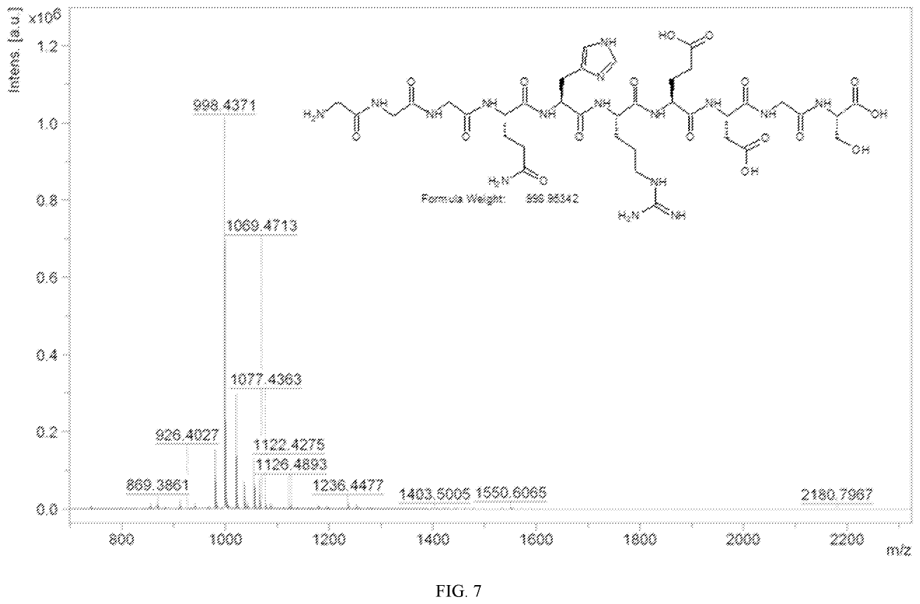

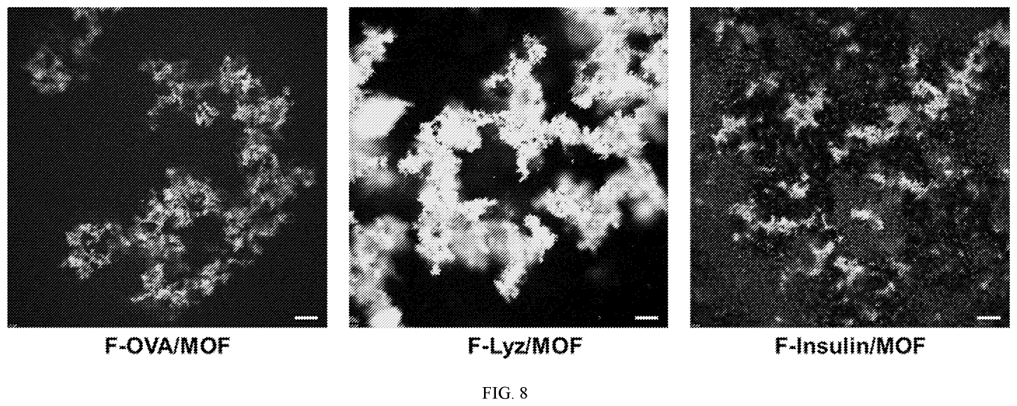

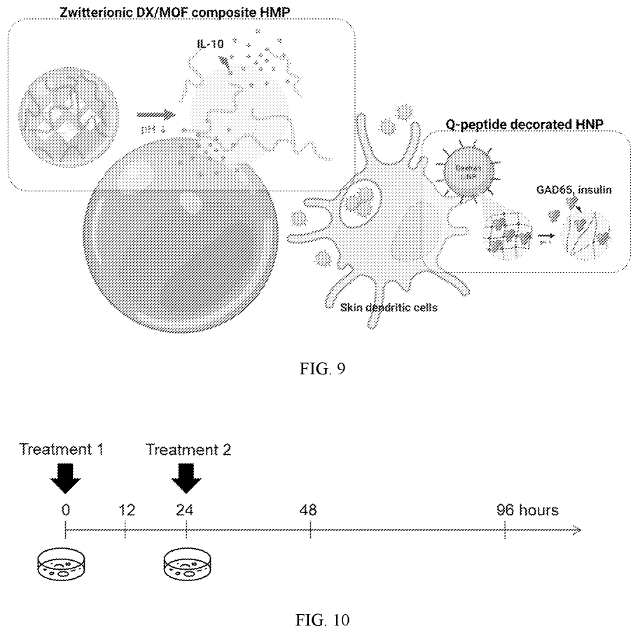

Angiopoietin-1-derived peptide (QHREDGS-NH₂: SEQ ID NO: 2) was selected to decorate the surface of NPs and the immunomodulatory effects were investigated. Tri-glycine bridge, GGG was added at the N-terminus of QHREDGS (SEQ ID NO: 2) (or Q-peptide) to increase the flexibility of peptide-receptor interactions. Therefore, in this study, GGGQHREDGS (SEQ ID NO: 1) was made in solid phase peptide synthesis as previously reported. Briefly, rink amide resins were used for peptide extension and HBTU/HOBt/DIPEA were used as catalysts. Peptide was cleaved with TFA/EDT (95/5) cocktail and the product was precipitated in cold ether. The product identity was verified with MALDI-TOF, as shown in FIG. 7. The peptide was grafted on NPs at the N-terminus to consume the excess aldehyde groups of NP solids in methanol to form imine bonds. Finally, the product tL-NPs were washed in extensively in methanol to remove unreacted peptides and kept in absolute ethanol for storage and sterilization.

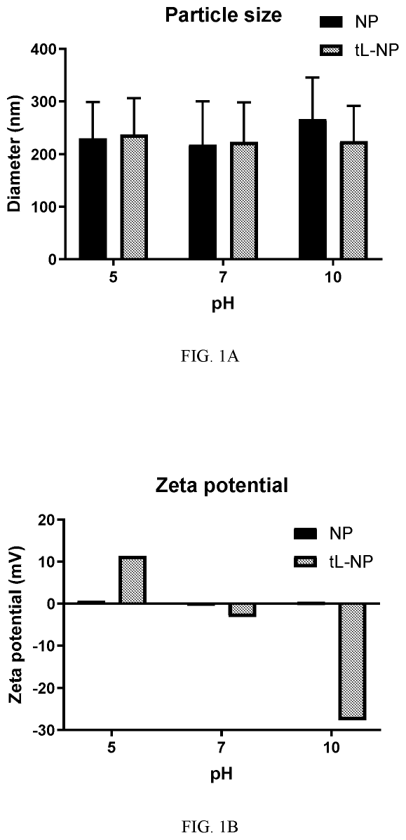

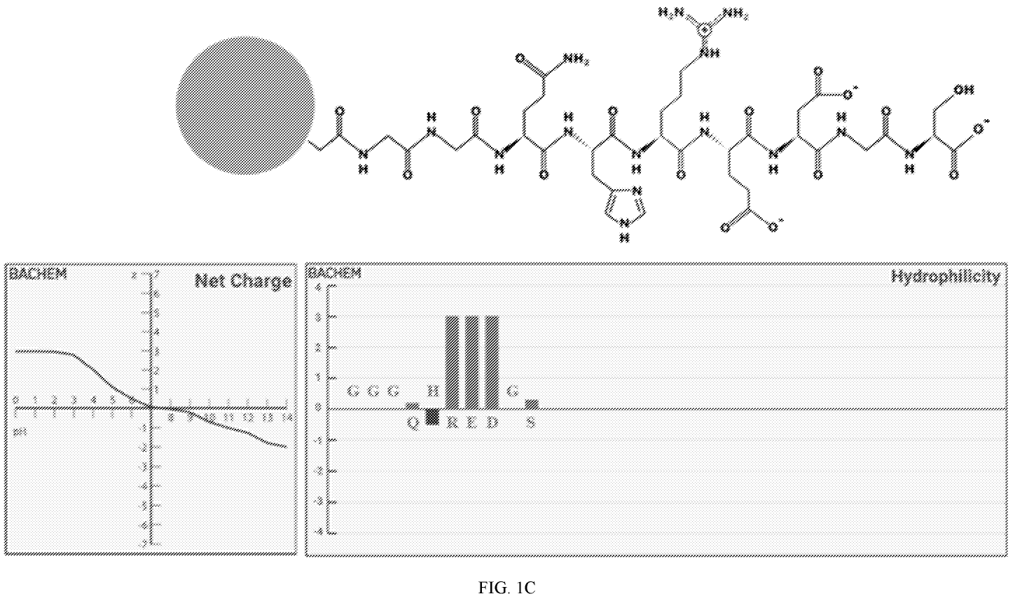

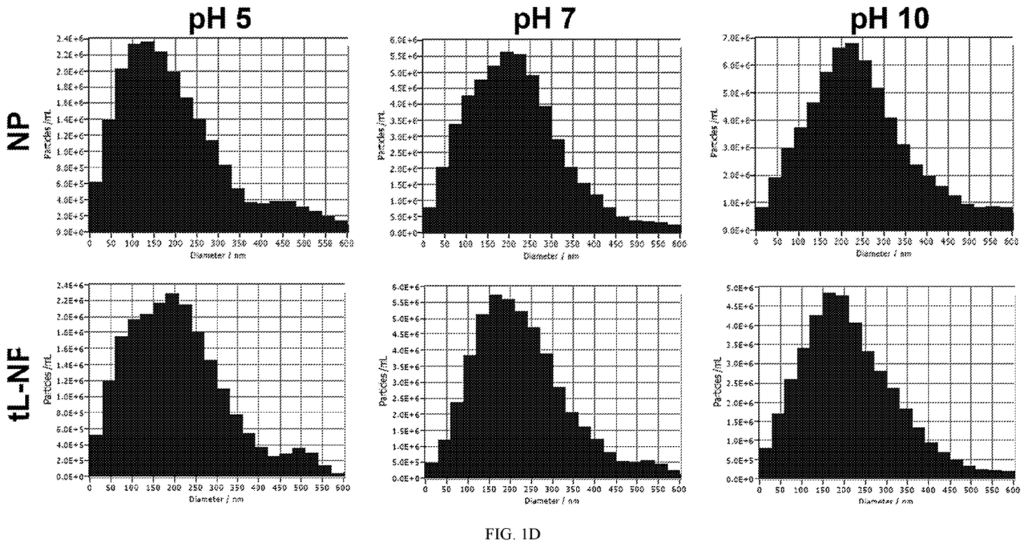

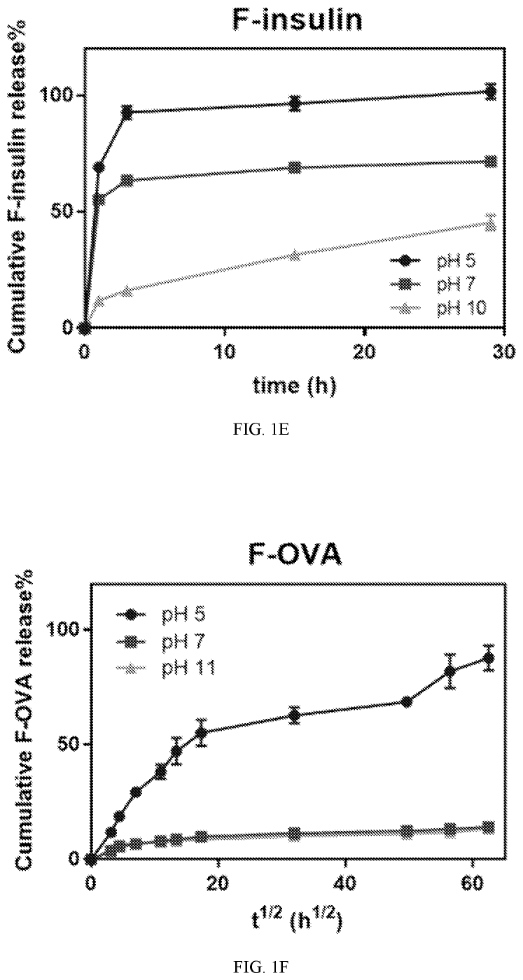

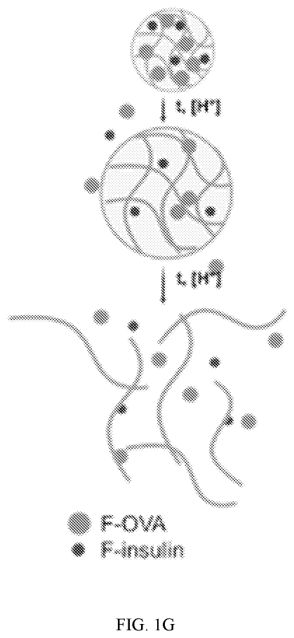

Both NP and tL-NP demonstrated similar initial size at the first 10 min incubation in water at various pH. In FIG. 1A, NP had an average diameter of 229±69.5 nm, 218±82.3 nm, and 266±79.5 nm in pH 5, 7 and 10 respectively. tL-NP also had the sizes of 237.3±66.8 nm, 233.4±74.8 nm, and 224.9±66.8 nm in the corresponding pH. The initial size distribution of NP and tL-NP are as indicated in FIG. 1D. Furthermore, the surface charge of particles was also measured with Zeta View NTA (DLS method). We observed that NPs were neutral at all pH, whereas tL-NP was cationic (+11.3 mV) at pH 5, −3.1 mV at pH 7 and −27.7 mV at pH 10. The changes in surface charge of tL-NPs were associated with the isoelectric point (pI) of the peptide coated on NPs. As shown in FIG. 1C, the theoretical pI of t-peptide was predicted to be 7.0 and the net charge decreases as pH increases.

The release profile of protein cargoes from NP were also probed with FITC-tagged insulin (F-insulin) and ovalbumin (F-OVA). OVA was used as model protein/antigen for GAD65 (65 kDa). Same dosage of proteins (0.25 mg/mL) were prepared in 30% wt/V polymer concentrations of DX-aldehyde and DX-carbazate (mixed at volume ratio 1:1) for both insulin-laden NP and OVA-laden NP. NPs were incubated in PB buffer of various pH, and the amount released was monitored with time. In FIG. 1E, F-insulin experienced rapid burst release at the initial time points. Almost 90% of total encapsulated F-insulin was released from NP within the first 6-hour at pH 5, 62% at pH 7 and 16% at pH 10. Conversely, F-OVA experienced slower release than F-insulin with only 55% of total encapsulated protein released at pH 5, 9% at pH 7 and 8.5% at pH 11. Such difference in release rate is due to the size difference between F-insulin and F-OVA, which is measured at 6 kDa and 45 kDa respectively. Moreover, F-OVA contains more lysine (20 residues) than F-insulin (1 residue), giving more reactive sites for the reaction between primary amine and aldehyde in forming imine bonds [24]. Therefore, the small insulin with less tethering sites could diffuse out from NP networks more readily than OVA. Furthermore, NPs experienced more rapid release of proteins in pH 5 than pH 7 due to the rapid hydrolysis of acid-sensitive hydrazone crosslinks [25], as depicted in FIG. 1G.

### EXAMPLE 2

### ZDX/MOF Physical Characterizations

We successfully prepared zDX/MOF composite hydrogels by first synthesizing ZIF-8/proteins MOF particles, then the dried solids were dispersed in zDX precursors dissolved in PBS, pH 7.4. Subsequently, hydrogel microparticles (HMPs) were formed in water-in-oil bulk emulsions, of which zDX polymers were crosslinked with DTT that had been pre- dissolved in oil phase, via thiol-Michael addition. The protein encapsulation efficiency for FITC-labelled lysozyme (F-Lyz) was approximately 90%.

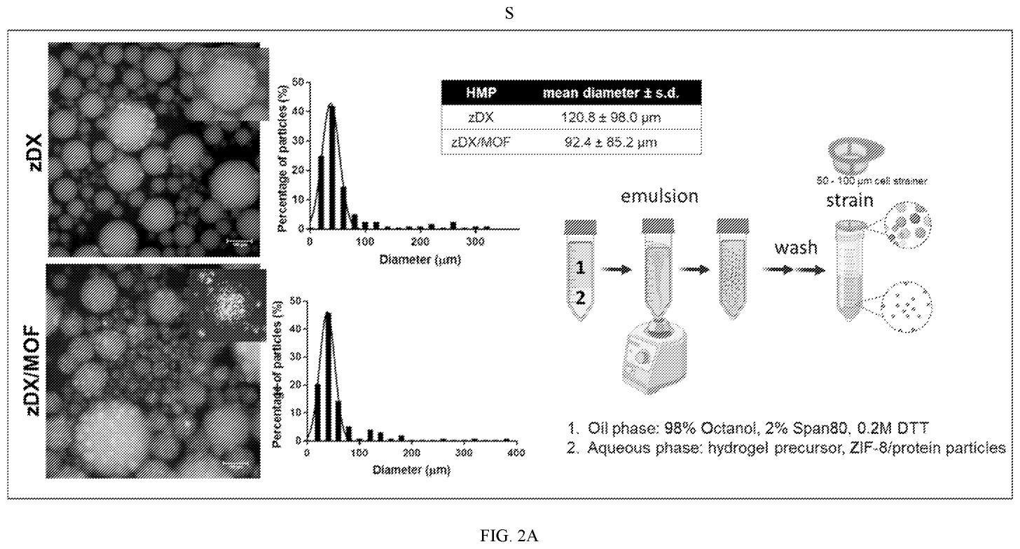

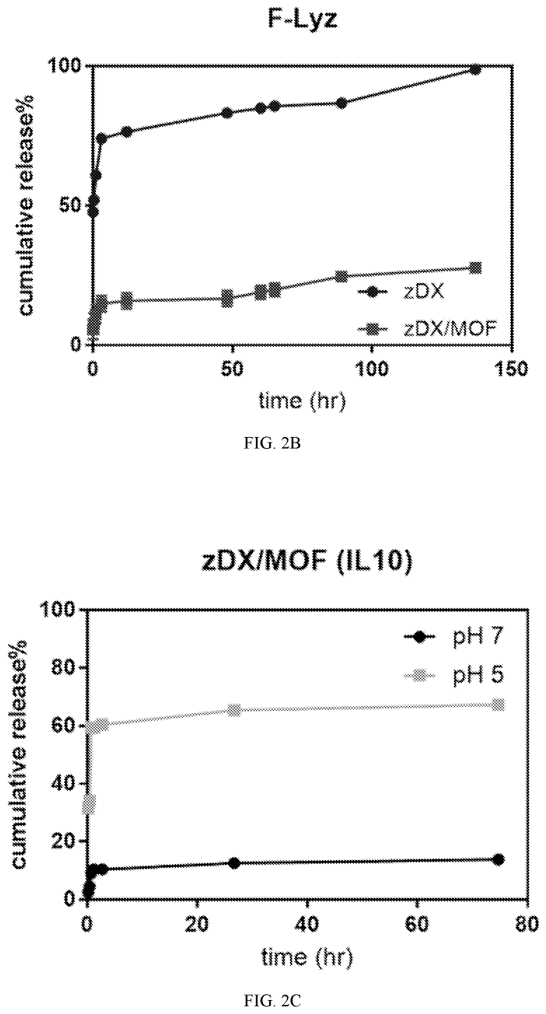

The morphology of zDX/MOF composite and zDX HMPs are as shown in FIG. 2A. The distribution of F-Lyz was significantly different in both HMPs. In contrast to zDX encapsulating F-Lyz, aggregates of ZIF-8/F-Lyz were visible within zDX/MOF microspheres. zDX and zDX/MOF had similar mean diameter. Nonetheless, bulk emulsion method gave rather large s.d. The crude HMPs were strained with 50 and 100 μm cell strainer to obtain HMPs with size range of 70 to 90 μm.

The release profile of zDX/MOF HMP was also probed with F-Lyz and IL-10. Both HMPs were formed at 10% wt/V polymer concentration, and DM of-OAc was approximately 4%. We observed that zDX gave rapid protein release with almost 75% of total encapsulated protein was liberated by 3-hour post-incubation in PBS, pH 7.4 at 37° C. Conversely, the incorporation of MOF could significantly suppress the protein release to 18% only in FIG. 2B. By day 5, almost 96% F-Lyz was recovered from zDX but only 28% of protein was released from zDX/MOF composites. Thus, the incorporation of MOF in hydrogel could effectively store and delay the release of small proteins. Next, we investigated the effect of pH on the protein release profile of zDX/MOF. The composite HMPs were incubated in PB buffer of 7 or 5 at 37° C. In FIG. 2C, IL-10 (18 kDa, pI 8) experienced much faster release at lower pH. Almost 60% of protein was released by 3-hour in pH 5; whereas at pH 7, HMPs only liberated 12% of total encapsulated protein. Indeed, ZIF-8/protein structure was sensitive to acid degradation. Study demonstrated that pH change from 7.4 to 6 in PBS was sufficient to release the encapsulated proteins in ZIF-8 nanoparticles [19]. We speculated that with the increase of H⁺ concentration, the, 2-methyl imidazole (pKa=7.85) would be protonated, causing the structure of ZIF-8 to dissociate and release the protein. Furthermore, the change in hydrogel microenvironment could perhaps influence the hydrolytic rate of hydrogel crosslinks within zDX/MOF, causing the HMPs to degrade faster.

### EXAMPLE 3

### Dual Spheres Effect on Ova Uptake and Intracellular Distributions in JAWSII

We proceeded to evaluate the performance of the above-mentioned dual hydrogel spheres system in generating potent tolerogenic DCs in vitro. F-OVA was used to probe the antigen uptake, intracellular trafficking, and presentation efficiency by JAWSII, an immature DC line. Herein, dual spheres were co-cultured with DC for 24 hours and the control groups such as untreated DC, single antigen-laden sphere, and bolus equivalent dose were included.

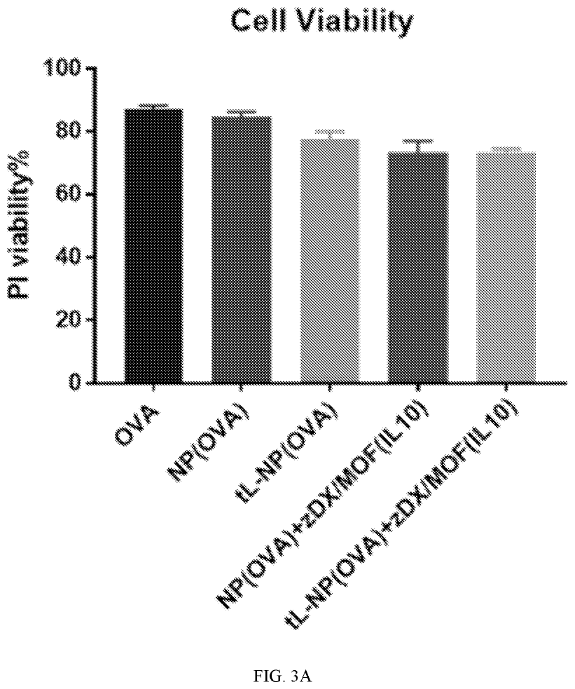

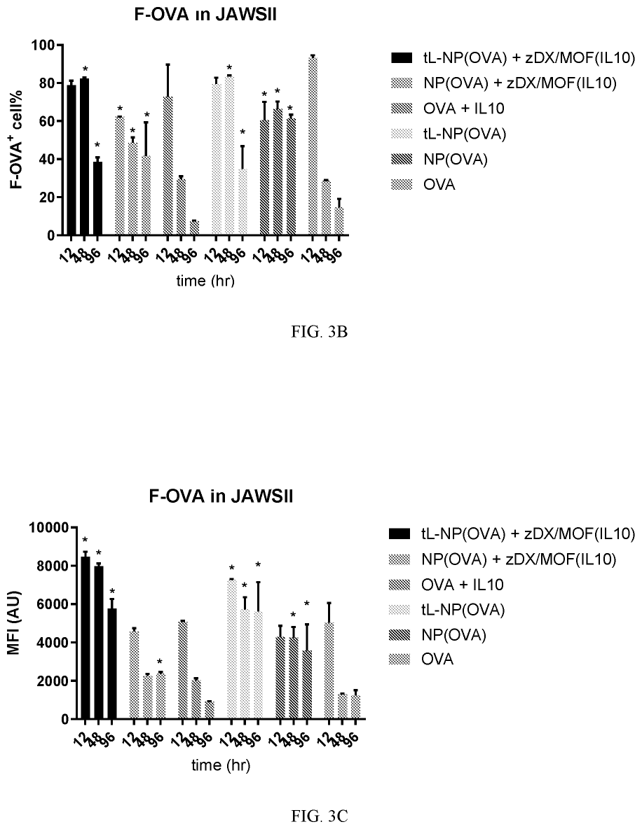

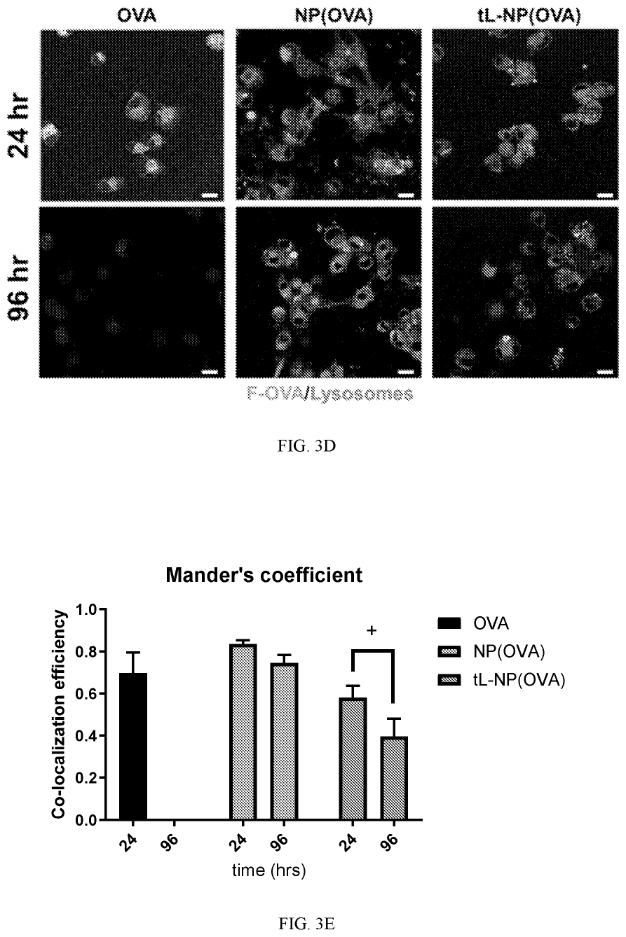

To investigate the ability of ligand-decorated NPs in promoting cellular uptake and intracellular storage, the cell cultures were washed with 0.001% Triton-X100 to remove un-internalized antigens at 24 hours post-treatment. Centrifugation at 500 g, 5 min was applied to remove NPs and supernatant while preserving the MPs with the cell pellets. The pellets were replated and observed for another 72 hours. The treatment schedule is summarized in Table 2 and FIG. 10. The cell viability was evaluated with propidium iodide exclusion staining and flow cytometry analysis. In FIG. 3A, the cells at 96-hour post-treatment exhibit viability of 70 to 80% for all experiment groups, justifying the biocompatibility of single (NP, tL-NP) and dual-spheres (NP+zDX/MOF, tL-NP+zDX/MOF) system.

TABLE 2
The treatment schedule of dual sized sphere
based T1D vaccines on JAWSII in vitro.

| Groups/treatment | 0 h | 24 h |
|---|---|---|
| OVA | 50 μg/mL OVA | Remove medium via |
| OVA + IL-10 | 50 μg/mL OVA + 10 ng/mL IL-10 | centrifugation (500 g, 5 |
| NP(OVA) | 0.1 mg/mL (~10⁸ NP particles, 100:1 | min), wash with 0.001% |
| tL-NP(OVA) | particle to cell ratio), ~50 μg/mL OVA | Triton-X, replace with fresh |
| NP(OVA) + | 0.1 mg/mL (~10⁸ NP particles, 100:1 | complete medium |
| zDX/MOF(IL-10) | particle to cell ratio), ~50 μg/mL OVA + | |
| tL-NP(OVA) + | 1.5 mg/mal (~10⁴ particles, 1:100 | |
| zDX/MOF(IL-10) | particle to cell ratio), ~50 ng/mL IL-10 | |

The decoration of NPs with Q-peptide was shown to enhance the antigen uptake by JAWSII. In FIG. 3B and C, tL-NP showed ˜79% F-OVA+cells and >7000 AU MFI as compared to NP which had lower uptake efficiency of ˜60% antigen positive cells and ˜4000 AU MFI. Furthermore, we observed that both tL-NP and NPs could prolong the intracellular antigen signals. After washing to remove un-internalized F-OVA, OVA, and OVA+IL-10 experienced drastic decrease of antigen signals with only ˜30% and ˜10% F-OVA⁺ signals at 48 and 96-hour post-treatment. The mean fluorescence intensity (MFI) of F-OVA in total live cell population also exhibited similar trend. In contrast, tL-NP and NP showed ˜79.7% and 63% F-OVA⁺ cells respectively at 48-hour. Nonetheless, the positive cells in tL-NP fell to ˜38.3% with extended culture while the level was maintained at ˜60% for NP group. We also observed that IL-10 in soluble form or loaded in zDX/MOF had no effect on the antigen uptake level and retention in JAWSII, which had been co-treated with soluble OVA or OVA-laden tL-NP. As shown in FIG. 3B and C, tL-NP(OVA)+zDX/MOF (IL-10) and OVA exhibited similar antigen signals profile as tL-NP(OVA) and OVA respectively. Nonetheless, the presence of IL-10 influenced the antigen retention by NP. In FIG. 3C, NP(OVA)+zDX/MOF (IL-10) showed significantly lower MFI of F-OVA in total cell populations at 48 and 96-hour than NP(OVA).

In addition, we investigated the intracellular distribution of F-OVA in FIG. 3D. JAWSII was treated with bolus OVA, OVA-laden NP, or tL-NP for 24 hours, then washed with 0.001% Triton-X100 to remove un-internalized antigens and particles. The antigen distribution within the cells was monitored at 24-and 96-hour post-treatment and the cells was stained with Lysotracker-Red to visualize the late endosome and lysosome compartments in FIG. 3D. The overlap coefficient was quantified as indicated in FIG. 3E. We observed co-localization (yellow) of F-OVA (green) and lysosomes (red) for all groups at 24-hour post-treatment. Nonetheless, the signals co-localization significantly reduced with extended culture in tL-NP(OVA) group, suggesting possible endosomal escapes of F-OVA mediated by the nanocarriers. In fact, tL-NP exhibited positive charge of +10 mV at pH 5 from the zeta potential measurements in FIG. 1B. It was speculated that the nanocarriers exerted proton sponge effect and as tL-NP degraded and swelled, the endosome structure would be compromised and resulted in the release the protein cargoes to the cytosol [26][27].

### EXAMPLE 4

### JAWSII Maturation Profile

Flow cytometry and ELISA was performed to evaluate JAWSII differentiation into immunogenic or tolerogenic phenotype. The phenotype was evaluated by the expression of known immunogenic markers CD86, CD40 and MHC-II and the secretion of pro-inflammatory cytokines IL-6 and TNF-α. Conversely, tolerogenic DCs were characterized by low expression level of co-stimulatory molecules, CD86 and CD40, and production of anti-inflammatory cytokine, IL-10 [28]-[30]. To simplify the comparison of DC activation states, the mean fluorescence intensity of given surface marker measured with flow cytometry was normalized by the expression of the non-treated DC.

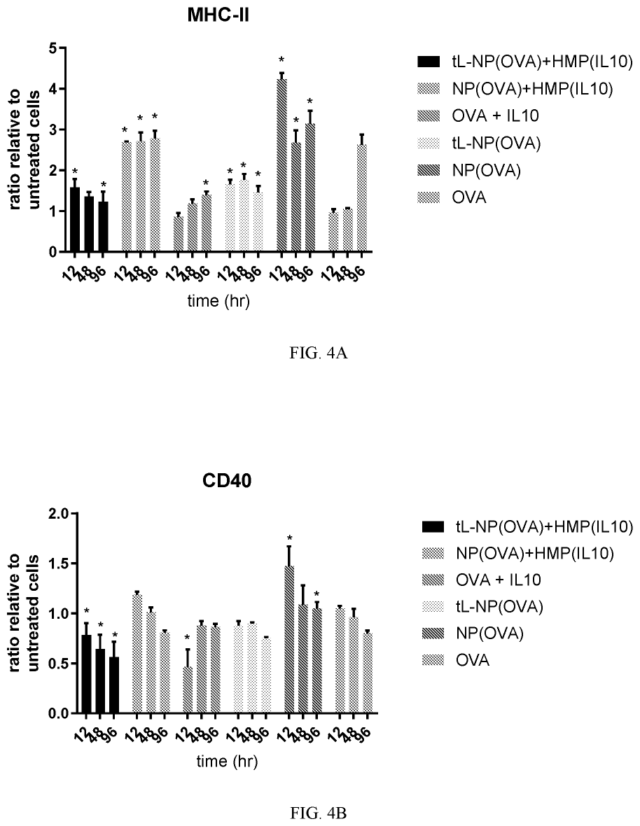

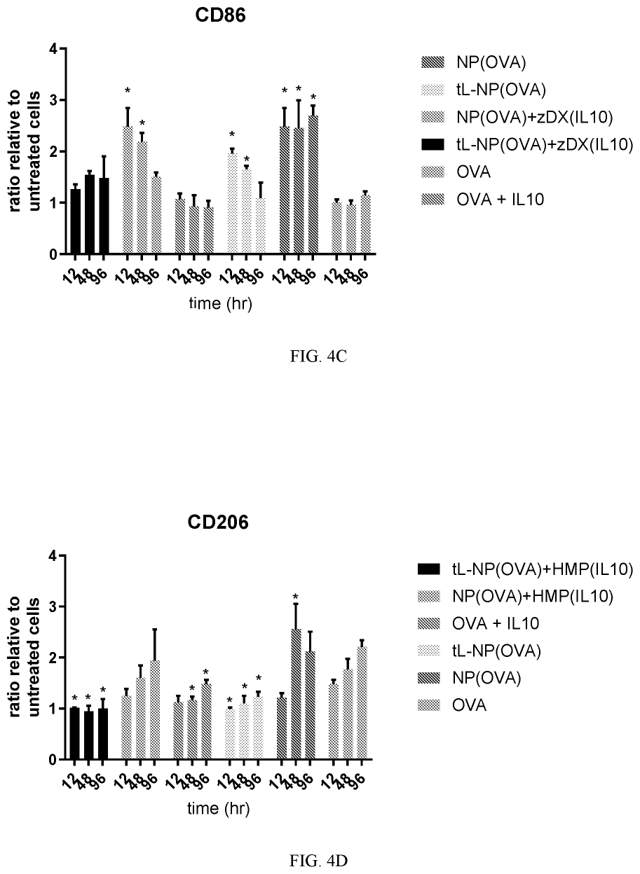

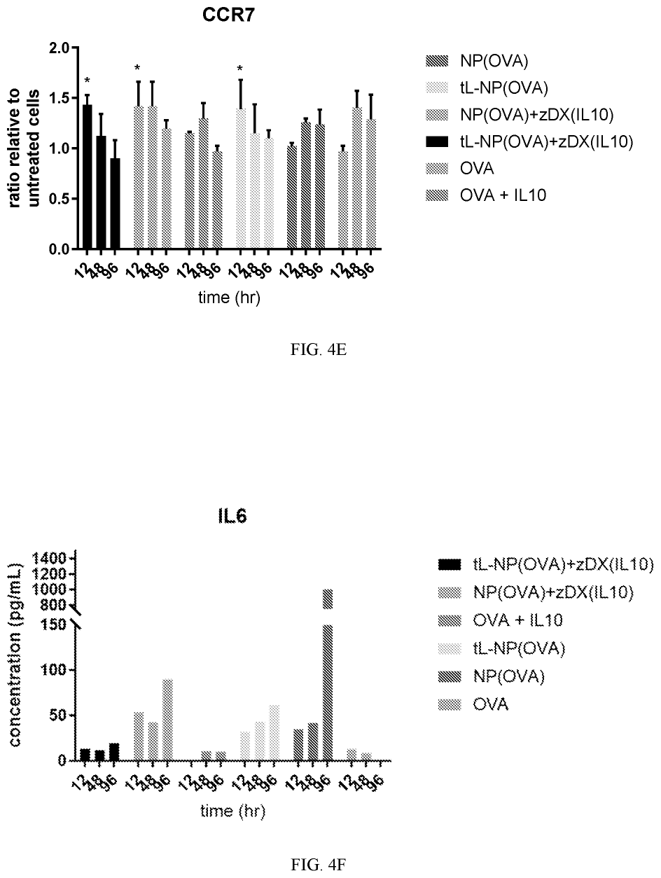

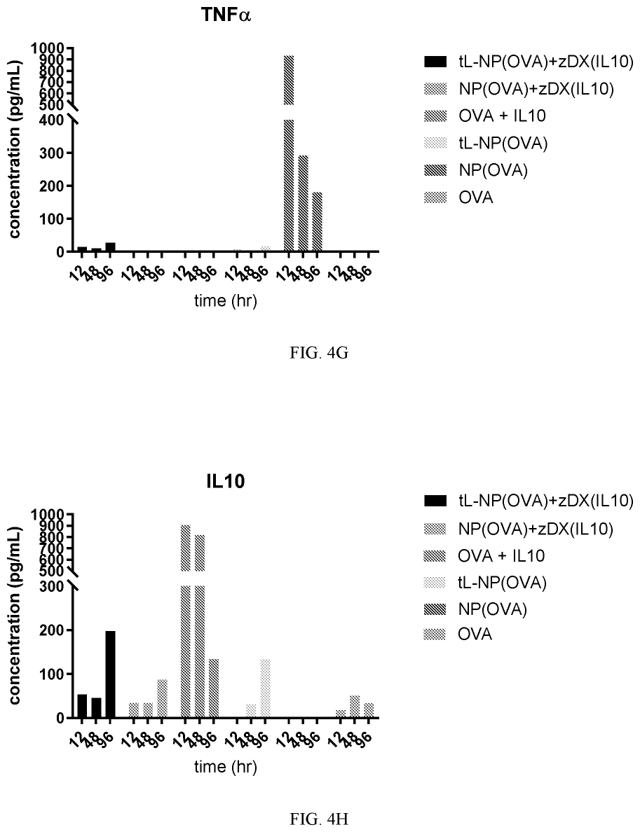

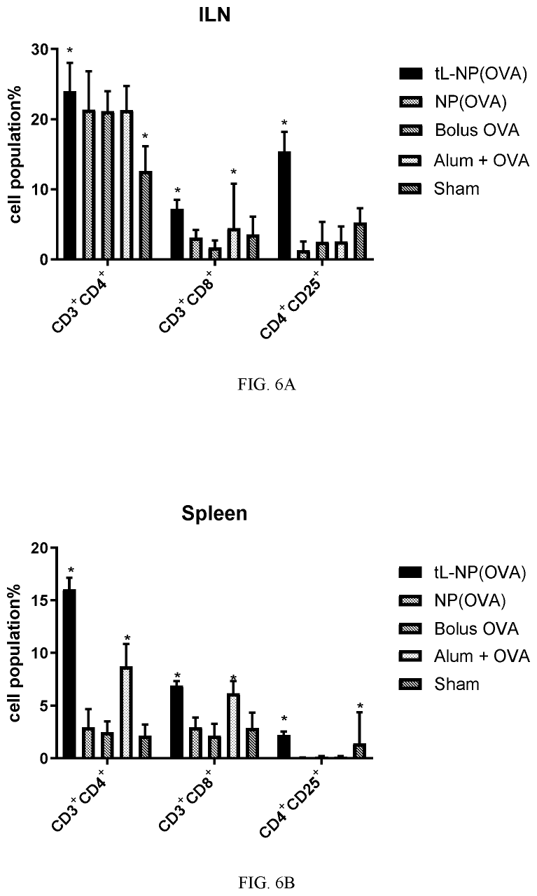

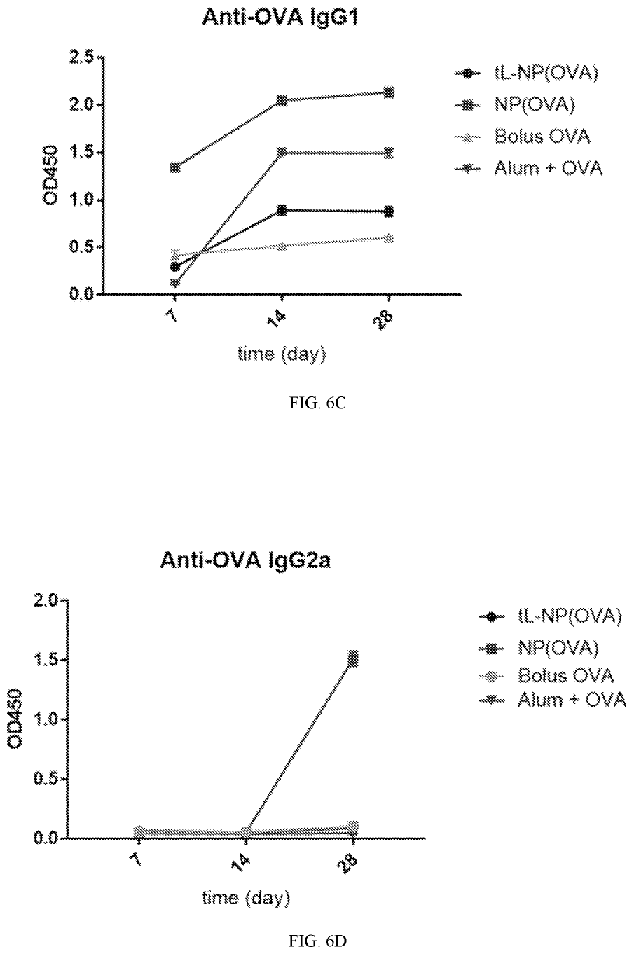

In FIGS. 4A-4C, NP(OVA) induced higher degree of immunogenic maturation of JAWSII with significantly greater expression level of MHC-II, CD86 and CD40. Moreover, the treatment also triggered cells to produce higher level of pro-inflammatory cytokines, IL-6 and TNF-α and IL-10 level was negligible in FIGS. 4F-4H. Conversely, tL-NP(OVA) was favourable in inducing tolerogenic DCs. The levels of MHC-II, CD40 and CD86 were persistently lower than NP(OVA). Notably, the expression of co-stimulatory molecules was similar to that of bolus OVA treatment. Moreover, the surface grafting of Q-peptide was also able to persistently suppress the production of pro-inflammatory cytokines, IL-6 to ˜50 pg/mL and TNF-α to ˜10 pg/mL, in contrast to NP(OVA) which induced >1000 pg/mL IL-6 at 96-hour and >900 pg/mL TNF-α at 12-hour post-treatment. Besides that, DCs produced ˜150 pg/mL IL-10 at 96-hour post-treatment with tL-NP, implicating the tolerogenic tendency of Q-peptide. Other studies have also demonstrated the immunomodulatory properties of Q-peptide on various delivery platforms. According to the reports, the immobilization of Q-peptide could induce macrophages to produce higher level of both pro- and anti-inflammatory cytokines to promote wound healing. Notably, Q-peptide film was able to induce macrophages to produce higher level of IL-10 and TGF-β; nonetheless, in terms of surface marker expression, the macrophages exhibited higher CD86 and lower CD206 levels [31]. This in consistent with our results, where tL-NP exhibited similar trend, i.e., higher CD86, less CD206 in FIG. 6C and FIG. 6D as compared to bolus OVA treated cells.

The co-incubation of antigen-laden nanospheres with bolus IL-10 and IL-10-laden zDX/MOF microspheres also exhibited DC modulating responses. In consistent with the immunosuppressing effect of soluble IL-10 on DC, the low level of cytokine released from zDX/MOF at initial timepoints was sufficient to inhibit the expression levels of MHC-II, CD40 and CD86 induced by NP(OVA). Moreover, the cytokine release could also further downregulate the expression level of CD40 and CD86 in tL-NP(OVA)+zDX/MOF (IL-10) co-culture. In terms of cytokine profile, the presence of IL-10 could also further suppress the generation of IL-6 and TNF-α triggered by the nanocarriers at the initial timepoints, as shown in FIG. 4F and FIG. 4G. Indeed, other reports have revealed that IL-10 could inhibit DC maturation induced by different endogenous and exogenous stimuli [31]. The cytokine also suppressed the ability of APCs to present antigens [32]. Additionally, we found that soluble IL-10 added to the medium could induce autocrine generation of the anti-inflammatory cytokine by DC. For OVA+IL-10 group, the cell was treated with 10 ng/ml soluble cytokine for 24 hours and followed by washing and replacement of fresh medium containing no IL-10. However, 812 pg/mL IL-10 was detected in the culture medium post-washing at 48-hour and 135 pg/mL at 96-hour. More importantly, our results indicated that zDX/MOF shows no effect in inducing DC maturation; therefore, the microcarrier was compatible with the immunosuppressive effect of IL-10.

We also investigated the effect of dual sphere system on the expression of CD206 and CCR7. CD206, also called mannose receptor, is a C-type lectin receptor which plays crucial role in mediating phagocytosis and endocytosis of pathogens and exogenous particles in DC. Its ligands include wide range of polysaccharides and lectins, such as dextran, mannan and glycoproteins [33][34]. In FIG. 4D, we showed that DX-based NP(OVA) could induce significant upregulation of CD206 to facilitate the uptake of particles. Nonetheless, the effect was significantly suppressed by IL-10 released from microspheres as well as Q-peptide grated on NPs in tL-NP groups. Despite of the downregulation of receptors, tL-NP(OVA) and tL-NP(OVA)+zDX/MOF (IL-10) showed higher level of antigen uptake in DC, as described in FIG. 3B and FIG. 3C. The results suggested that Q-peptide may facilitate the enhanced antigen uptake via different pathways in DC, possibly regulated by integrins.

On the other hand, CCR7 signifies the lymph node homing marker in DC. We observed that CCR7 level was downregulated with the addition of soluble OVA. This result agreed with other studies which also reported the inhibitive role of IL-10 on CCR7 expression [31]. On the other hand, the zDX/MOF microsphere system could afford low level of CCR7 released at the initial timepoints, thereby allowing transiently higher expression of CCR7, as indicated in FIG. 6E. However, the marker level fell with increasing amount of IL-10 accumulated in the medium. On the other hand, we observed that the decoration of Q-peptide on tL-NPs was sufficient to induce higher level of CCR7 at initial timepoint, but the addition of IL-10-laden zDX/MOF exhibited no added effect on the chemokine receptor expression, as shown in FIG. 4E.

### EXAMPLE 5

### Biodistribution of OVA Delivered In TL-NP

Next, we evaluated the effect of Q-peptide in mediating the biodistribution of OVA-laden NPs. Animals were injected subcutaneously with same dosage of IR-labelled OVA (500 μg/animal with an average weight of 22 g) in various forms, tL-NP, NP, bolus, and alum. Alum is a commercial adjuvant in various vaccine formulations; thus, it was used as positive control in this study. On the other hand, animals in sham group were treated with PBS, and the organs were used as blank to define the signal intensity threshold in IR measurements. The antigen distributions in skin at injection site, inguinal lymph node (ILN), spleen, liver, kidneys, and lungs were monitored on day 7, 14 and 21. Animals were sacrificed at the different timepoints, and the organs were collected to measure antigen signal intensity with an IR scanner.

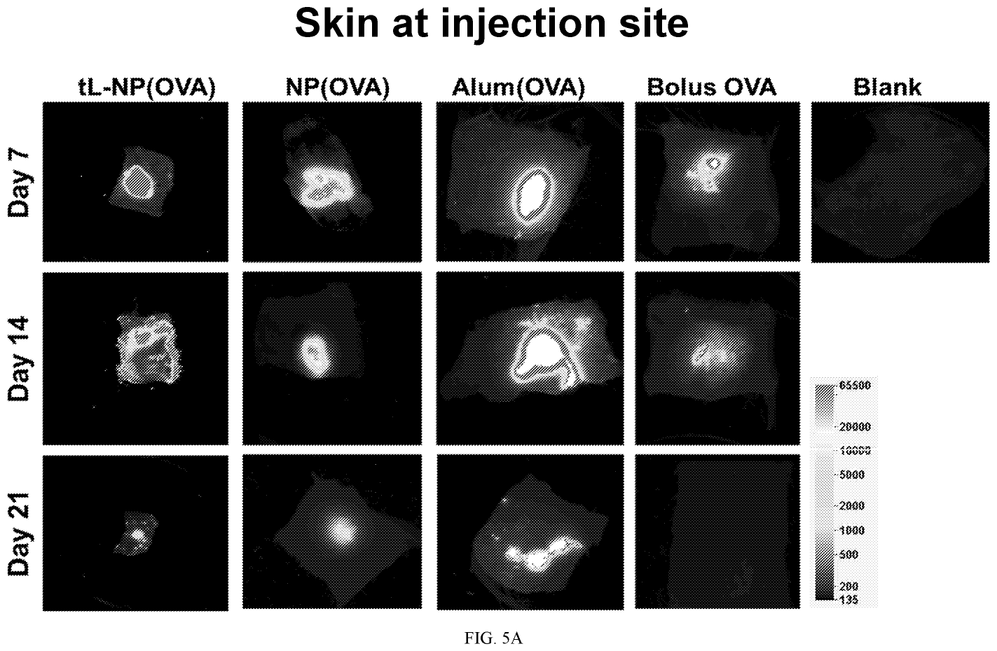

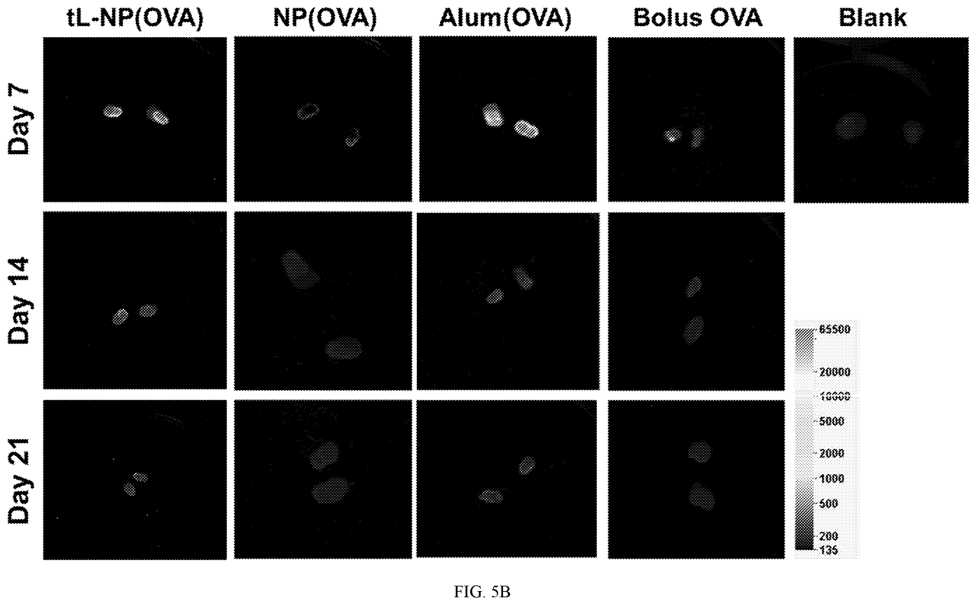

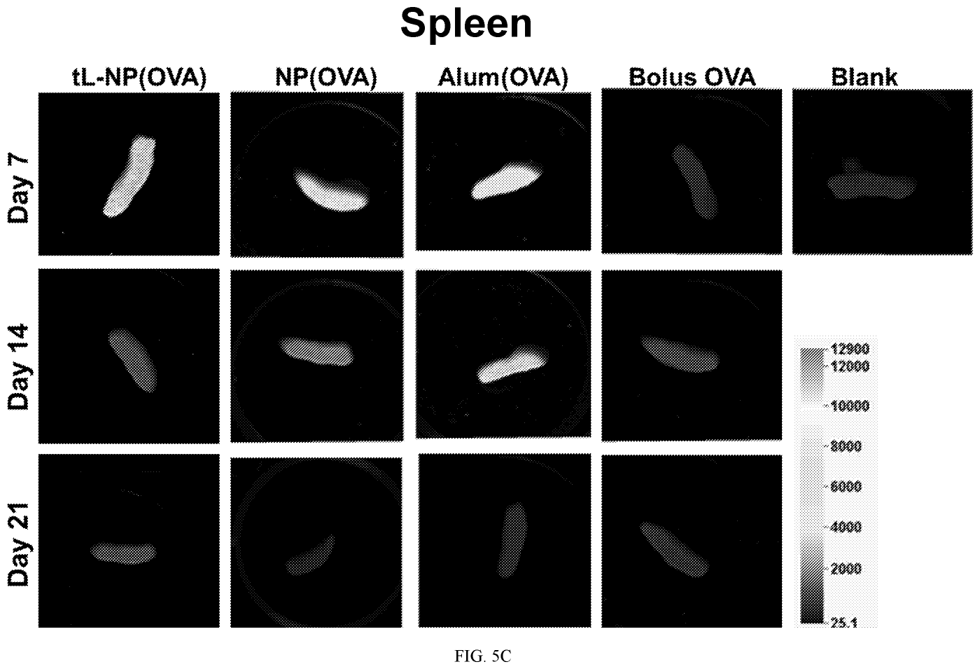

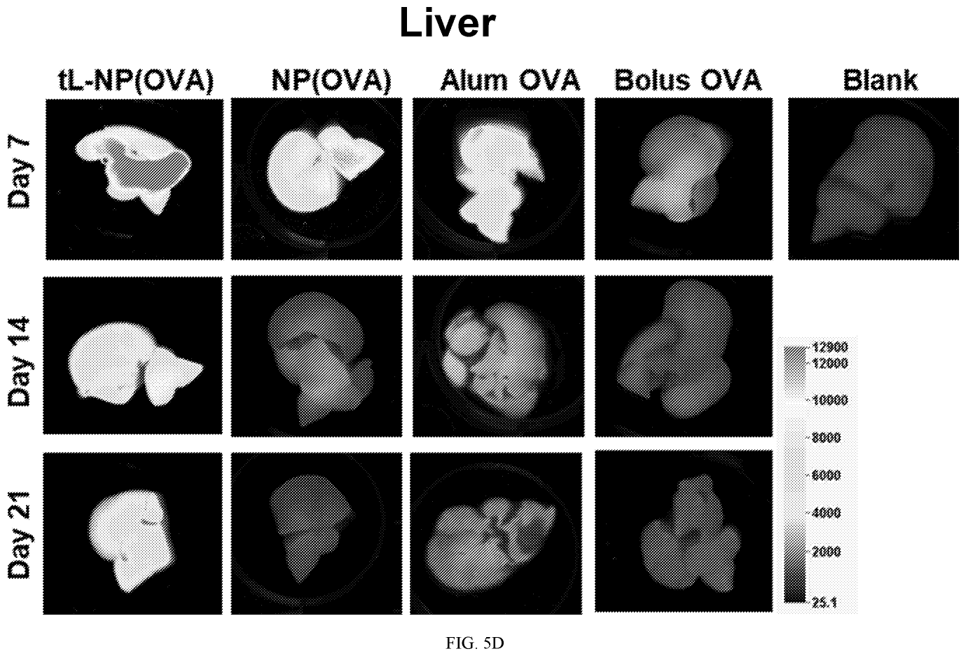

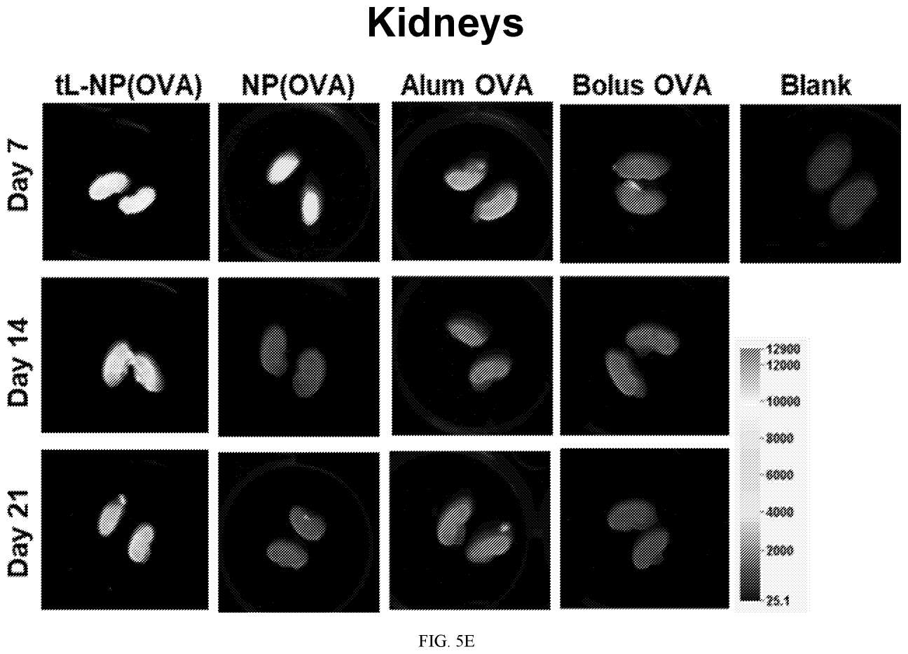

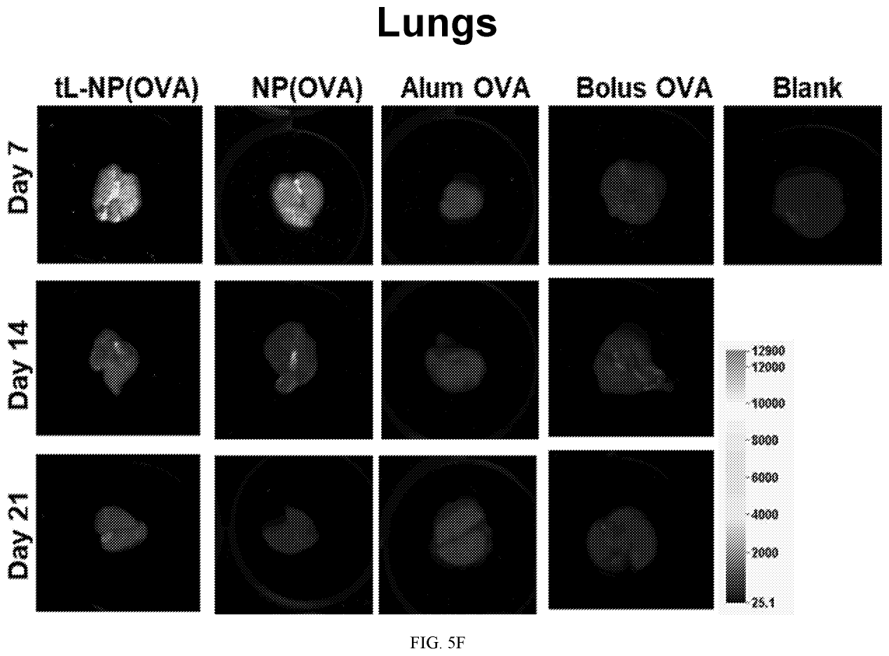

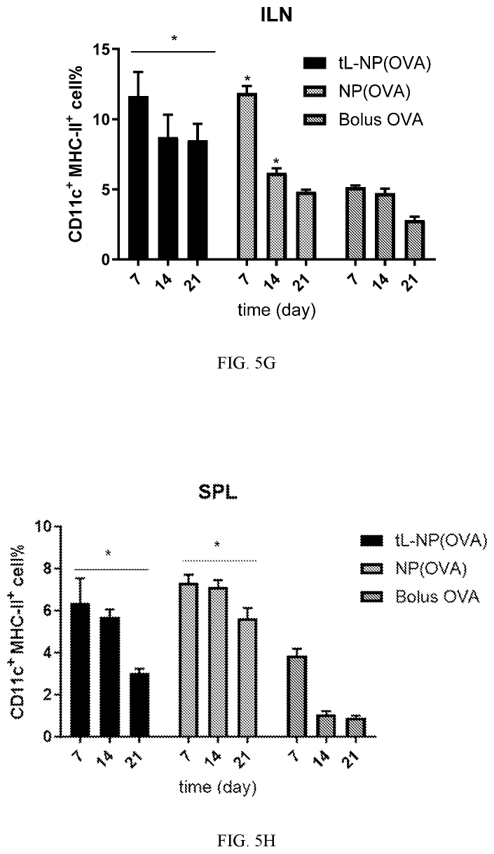

As shown in FIG. 5A, both tL-NP and NP could give higher antigen retention rate than bolus OVA at the injection site up to 3 weeks post-injection. In addition, the signal intensity was higher for tL-NP than NP, but lower than alum OVA on day 7 and 14. The biodistribution and excretion rate of particles largely depend on the size and surface chemistry. Alum forms micron-sized depot to extend antigen retention in the subcutaneous region, whereas NPs with size ranged 200 to 300 nm in diameter were cleared to the systemic circulation [35][36]. In FIGS. 5D-5F, we observed tL-NPs could retain higher overall antigen signals than NPs and bolus OVA in the body for longer period of time. Antigen signals could be detected in liver, kidney, and lungs up to 3 weeks post-injection. In fact, these organs could generate endogenous Angpt-1 and interact with paracrine receptors to maintain organ functions. For instance, Angpt-1 plays key role in maintaining vessel health and remodelling in vascular rich networks in liver, kidneys, and lungs. Notably, Angpt-1-Tie1/2 interactions mediate the barrier functions of glomerular filtration complex, and abnormalities implicated

diabetic kidney disease [37], [38]. Herein, we speculated that Q-peptide confers the interaction between tL-NPs and the resident cells expressing the target receptors in liver, kidneys, and lungs, thereby providing local enrichment of cargoes for an extended period of time. Such engagement could be leveraged by the nanocarriers to provide additional therapeutic advantages in protecting the organs of T1D patients.

Upon activation, peripheral DCs are able to migrate to secondary lymphoid organs, such as lymph nodes and spleen via CCR7-CCL19/21 axis, bringing the processed antigens to the T-cell rich zones for antigen presentation to activate naïve T-cells. Motivated by the established in vitro results on CCR7 response of DC, we proceeded to investigate the migration rate of activated skin DCs by monitoring the changes in the antigen signal intensity in inguinal lymph nodes (ILN) and spleen. We also analysed the population frequency of CD11c⁺ MHC-II⁺ cells, implicating mature DCs present in the secondary lymphoid organs. As shown in FIG. 5B, OVA-laden tL-NP and alum showed higher level of antigen signals in ILN than NP and bolus OVA on day 7 and 14. The results are in consistent with the in vitro CCR7 expression analysis, whereby tL-NP could induce significantly higher level of surface marker than NP in FIG. 4E. Moreover, in FIG. 5G, the population frequency of CD11c⁺ MHC-II⁺ was higher in tL-NP than NP and bolus OVA on day 7 and 14 post-injection. These results suggested that the antigens were actively trafficked from the skin injection site to the nearby ILN by CD11c⁺ MHC-II⁺ DC via the action mechanism of CCR7. Other studies also made similar observations on the antigen delivery by the migrating DCs [39][40] [41]. The possibility passive drainage of NPs to ILN was excluded in this study as the size of NPs (200 to 300 nm) were much larger than the requirements (5 to 100 nm) to enter the lymphatic capillaries [42]. Nonetheless, the antigen trafficking to spleen exhibited different trend, with higher antigen signals in Alum OVA and NP(OVA) on day 7 and 14 than tL-NP(OVA). Moreover, the population frequency of CD11c⁺ MHC-II⁺ cells were higher in NP(OVA) on day 14 and 21 than tL-NP(OVA), as shown in FIG. 5H.

### EXAMPLE 6

### OVA-Specific Cellular and Humoral Responses

The subcutaneous immunization with OVA as antigen could induce activation of CD4⁺ and CD8⁺ T-cells as shown in FIG. 6A and FIG. 6B. On day 7 post-injection, the T-cell population distributions in ILN and spleen were quantified with flow cytometry to identify CD3⁺CD4⁺ (T-helper cells), CD3⁺CD8⁺ (T-killer cells) and CD4⁺CD25⁺ (T-regulatory cells) populations. In the ILN, OVA immunization with different dosage forms could trigger significantly higher level of CD3⁺CD4⁺ cells. As shown in FIG. 6A, the cell population frequency was measured at 24%, 21.4%, 21%, and 21.3% for tL-NP(OVA), NP(OVA), alum+OVA and bolus OVA respectively, as compared to 12.6% in sham group. Moreover, tL-NP could also induce the highest level of CD4⁺CD25⁺ cells than amongst the treatment groups. The cell level was 15.4% as compared to NP(OVA) which only induced 4.17% and 4.45% in Alum+OVA. The results implicate that tL-NP(OVA) could be beneficial for suppressing auto-immune response against antigen cargo.

Nonetheless, different T-cell population profile was observed in spleen. In FIG. 6B, tL-NP induced the highest level of CD3⁺CD4⁺ cells, measured at 16.05% amongst other treatment groups, which only activated 2.9%, 8.7%, 2.4% and 2.1% for NP, alum, bolus, and sham respectively. Moreover, the level of CD3⁺CD8⁺ induced by tL-NP was similar to that of alum OVA. The overall higher level of CD8⁺ cells induced by tL-NP than NP could be due to the observations of endosomal escaping of antigens to the cytosol as shown in FIG. 3D and FIG. 3E and accessing the MHC-I processing machinery. Conversely, tL-NP(OVA) induced the highest level of CD4⁺CD25⁺ cells, at 2.18% amongst other treatment groups. On day 7, the induction of T-regulatory cells by alum OVA and NP(OVA) was negligible.

For the humoral response, we monitored the serum levels of OVA-specific IgG1 and IgG2a on day 7, 14 and 28 with ELISA. As shown in FIG. 6C, the adjuvanted formulations, NP, and alum could induce higher level of IgGI titre than the group which received bolus OVA only. Furthermore, in FIG. 6D, NP(OVA) could induce the highest level of IgG2a on day 21. Nonetheless, the decoration of Q-peptide on tL-NPs could significantly suppress both IgGI and IgG2a level induced by NP(OVA) on day 14 and 21.

Herein, we also measured the serum level of various cytokines on day 7 post-immunization to gauge the inflammatory responses of subcutaneous adjuvants. We observed that NP(OVA) induced stronger pro-inflammatory response with higher plasma IL-6 and TNF-α, measuring at 6.9 pg/mL and 107.4 pg/mL respectively. Table 3 also shows that tL-NP induced lower level of TNF-α at 68.1 pg/mL, while IL-6 was non-detectable. Conversely, both NP(OVA) and bolus OVA groups did not induce IL-10 on day 7, while tL-NP administration induced 51.7 pg/mL IL-10 in the blood plasma. The results implicate that tL-NPs is a promising tolerogenic adjuvant for the suppression of autoimmune disorders.

Indeed, tL-NP was grafted with Q-peptide, which is derived from Angpt-1. The anti-inflammatory roles of the protein have been demonstrated in several studies. It could help to maintain the barrier functions of endothelium and suppress inflammation-induced vascular leakage [43][44]. The direct immunomodulatory effects of Angpt-1 and its peptide derivatives have also been investigated on macrophages and monocytes. Unlike the whole-body response, cell culture studies revealed that Angpt-1 induced pro-inflammatory responses on macrophages with higher production level of TNF-α [45]. Nonetheless, the peptide derivative-QHREDGS (SEQ ID NO: 2) could induce macrophages to produce both pro-inflammatory (IL-1β, IFN-γ) and anti-inflammatory cytokines (TGF-β, IL-10 and IL-4) [31].

TABLE 3
The levels of serum cytokines, IL-6, TNF-α, IFN-γ
and IL-10 at 21 days post-immunization with different
vaccine formulations, NP(OVA), tL-NP(OVA) and bolus (OVA).

| | IL-6 | TNF-α | IFN-γ | IL-10 |
|---|---|---|---|---|
| NP(OVA) | 6.9 ± 1.6 | 107.4 ± 22.6 | n.d. | n.d. |
| tL-NP(OVA) | n.d. | 68.1 ± 5.2 | n.d. | 51.7 ± 24.0 |
| Bolus OVA | 5.3 ± 1.2 | 52.2 ± 24.1 | n.d. | n.d. |

N = 5, mean ± s.d. n.d. refers to non-detectable, of which the cytokines were below signal threshold of ELISA measurements.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EMBODIMENTS

Embodiment 1. A hydrogel comprising a nanoparticle, a microparticle, at least one peptide on the surface of the nanoparticle, and at least one zwitterionic motif on the surface of the microparticle, wherein each of the nanoparticle and the microparticle comprises a polysaccharide.
Embodiment 2. The hydrogel of embodiment 1, wherein the at least one polysaccharide is dextran.
Embodiment 3. The hydrogel of embodiment 1, wherein the at least one peptide comprises insulin or GAD65 or the amino acid sequence GGGQHREDGS (SEQ ID NO: 1) or QHREDGS (SEQ ID NO: 2).
Embodiment 4. The hydrogel of embodiment 1, wherein a mean diameter of the microparticle is at least about 50 μm.
Embodiment 5. The hydrogel of embodiment 1, wherein a mean diameter of the nanoparticle is about 100 nm to about 300 nm.
Embodiment 6. The hydrogel of embodiment 1, wherein the zwitterionic motif is a metal organic framework (MOF).
Embodiment 7. The hydrogel of embodiment 6, wherein the metal organic framework is zeolitic imidazolate framework (ZIF) 8.
Embodiment 8. The hydrogel of embodiment 1, wherein the zwitterionic motif further comprises a cytokine.
Embodiment 9. The hydrogel of embodiment 8, wherein the cytokine is IL-10.
Embodiment 10. A method for inducing tolerance differentiation of a dendritic cell of a subject, the method comprising administering an effective amount of the hydrogel of embodiment 1 to the subject.
Embodiment 11. The method of embodiment 10, wherein the dendritic cell is in the skin of the subject.
Embodiment 12. The method of embodiment 10, wherein the administering is a subcutaneous administration.
Embodiment 13. The method of embodiment 10, wherein the subject has Type-1 diabetes.
Embodiment 14. The method of embodiment 10, further comprising releasing the at least one peptide from the surface of the nanoparticle in the subject over a time period of at least about 6 hours.
Embodiment 15. The method of embodiment 10, wherein the zwitterionic motif further comprises a cytokine.
Embodiment 16. The method of embodiment 15, further comprising releasing the cytokine from the zwitterionic motif in the subject over a time period of at least about 6 hours.

### REFERENCES

[1] Bluestone, J. A., Buckner, J. H., & Herold, K. C., “Immunotherapy: Building a bridge to a cure for type 1 diabetes,” Science, vol. 373, no. 6554, pp. 510-516, July 2021.
[2] Di Lorenzo, T. P., Peakman, M., & Roep, B. O., “Translational mini-review series on type 1 diabetes: Systematic analysis of T cell epitopes in autoimmune diabetes,” Clin. Exp. Immunol., vol. 148, no. 1, pp. 1-16, April 2007.
[3] Nicholas, D., Odumosu, O., & Langridge, W. H. R., “Autoantigen Based Vaccines for Type 1 Diabetes,” Discov. Med., vol. 11, no. 59, p. 293, April 2011.
[4] Coppieters, K. & von Herrath, M., “Antigen-Specific Peptide Immunotherapy for Type 1 Diabetes: Proof of Safety, Hope for Efficacy,” Cell Metab., vol. 26, no. 4, pp. 595-597, October 2017.
[5] Lu, J., Liu, J., Li, L., Lan, Y., & Liang, Y., “Cytokines in type 1 diabetes: mechanisms of action and immunotherapeutic targets,” Clin. Transl. Immunol., vol. 9, no. 3, January 2020.
[6] Yoon, Y. M., Lewis, J. S., Carstens, M. R., Campbell-Thompson, M., Wasserfall, C. H., Atkinson, M. A., & Keselowsky, B. G., “A combination hydrogel microparticle-based vaccine prevents type 1 diabetes in non-obese diabetic mice,” Sci. Rep., vol. 5, August 2015.
[7] J, L., D, K., R, C., T, B., L, C., J, G., O, K., T, O., P, P., JJ, R., et al., “GAD65 antigen therapy in recently diagnosed type 1 diabetes mellitus,” N. Engl. J. Med., vol. 366, no. 5, pp. 433-442, February 2012.
[8] Sun, J., Shi, J., Li, J., Wu, M., Li, Y., Jia, S., Ma, C., Wang, X., Li, Z., Hu, N., et al., “The Effect of Immunosuppressive Adjuvant Kynurenine on Type 1 Diabetes Vaccine,” Front. Immunol., vol. 12, p. 2578 July 2021.
[9] Dénes, B., Fodor, I., & Langridge, W. H. R., “Autoantigens plus interleukin-10 suppress diabetes autoimmunity.,” Diabetes Technol. Ther., vol. 12, no. 8, pp. 649-661, 2010.
[10] Lewis, J. S., Dolgova, N. V., Zhang, Y., Xia, C. Q., Wasserfall, C. H., Atkinson, M. A., Clare-Salzler, M. J., & Keselowsky, B. G., “A combination dual-sized microparticle system modulates dendritic cells and prevents type 1 diabetes in prediabetic NOD mice,” Clin. Immunol., vol. 160, no. 1, pp. 90-102, September 2015.
[11] Husseiny, M. I., Du, W., Mbongue, J., Lenz, A., Rawson, J., Kandeel, F., & Ferreri, K., “Factors affecting Salmonella-based combination immunotherapy for prevention of type 1 diabetes in non-obese diabetic mice,” Vaccine, vol. 36, no. 52, pp. 8008-8018 December 2018.
[12] Muir, A., Peck, A., Clare-Salzler, M., Song, Y. H., Cornelius, J., Luchetta, R., Krischer, J., & Maclaren, N., “Insulin immunization of nonobese diabetic mice induces a protective insulitis characterized by diminished intraislet interferon-gamma transcription,” J. Clin. Invest., vol. 95, no. 2, pp. 628-634, 1995.
[13] Verbeke, C. S., Gordo, S., Schubert, D. A., Lewin, S. A., Desai, R. M., Dobbins, J., Wucherpfennig, K. W., & Mooney, D. J., “Multicomponent Injectable Hydrogels for Antigen-Specific Tolerogenic Immune Modulation,” Adv. Healthc. Mater., vol. 6, no. 6, pp. 1-28, 2017.
[14] Cifuentes-Rius, A., Desai, A., Yuen, D., Johnston, A. P. R., & Voelcker, N. H., “Inducing immune tolerance with dendritic cell-targeting nanomedicines,” Nat. Nanotechnol. 2020 161, vol. 16, no. 1, pp. 37-46, December 2020.
[15] Amodio, G. & Gregori, S., “Human tolerogenic DC-10: perspectives for clinical applications,” Transplant. Res., vol. 1, no. 1, p. 14, September 2012.
[16] Comi, M., Amodio, G., & Gregori, S., “Interleukin-10-Producing DC-10 Is a Unique Tool to Promote Tolerance Via Antigen-Specific T Regulatory Type 1 Cells,” Front. Immunol., vol. 9, no. APR, April 2018.
[17] Chung, J. T., Lau, C. M. L., & Chau, Y., “The effect of polysaccharide-based hydrogels on the response of antigen-presenting cell lines to immunomodulators,” Biomater. Sci., vol. 9, no. 19, pp. 6542-6554, 2021.
[18] Lau, C. M. L., Jahanmir, G., Yu, Y., & Chau, Y., “Controllable multi-phase protein release from in-situ hydrolyzable hydrogel,” J. Control. Release, 2021.
[19] Liang, K., Ricco, R., Doherty, C. M., Styles, M. J., Bell, S., Kirby, N., Mudie, S., Haylock, D., Hill, A. J., Doonan, C. J., et al., “Biomimetic mineralization of metal-organic frameworks as protective coatings for biomacromolecules,” Nat. Commun. 2015 61, vol. 6, no. 1, pp. 1-8, June 2015.
[20] Cai, H., Wu, F. Y., Wang, Q. L., Xu, P., Mou, F. F., Shao, S. J., Luo, Z. R., Zhu, J., Xuan, S. S., Lu, R., et al., “Self-assembling peptide modified with QHREDGS as a novel delivery system for mesenchymal stem cell transplantation after myocardial infarction,” FASEB J., vol. 33, no. 7, pp. 8306-8320 July 2019.
[21] Xiao, Y., Reis, L. A., Feric, N., Knee, E. J., Gu, J., Cao, S., Laschinger, C., Londono, C., Antolovich, J., McGuigan, A. P., et al., “Diabetic wound regeneration using peptide-modified hydrogels to target re-epithelialization,” Proc. Natl. Acad. Sci. U.S.A., vol. 113, no. 40, pp. E5792-E5801, October 2016.
[22] Dupuy, A. G. & Caron, E., “Integrin-dependent phagocytosis-spreading from microadhesion to new concepts,” J. Cell Sci., vol. 121, no. 11, pp. 1773-1783 June 2008.
[23] Li, H., Oliver, T., Jia, W., & He, Y. W., “Efficient dendritic cell priming of T lymphocytes depends on the extracellular matrix protein mindin,” EMBO J., vol. 25, no. 17, pp. 4097-4107 September 2006.
[24] Hermanson, G. T., “Vaccines and Immunogen Conjugates,” Bioconjugate Tech., pp. 839-865, January 2013.
[25] Zhang, Z., He, C., & Chen, X., “Hydrogels based on pH-responsive reversible carbon-nitrogen double-bond linkages for biomedical applications,” Mater. Chem. Front., vol. 2, no. 10, pp. 1765-1778 September 2018.
[26] Varkouhi, A. K., Scholte, M., Storm, G., & Haisma, H. J., “Endosomal escape pathways for delivery of biologicals,” J. Control. Release, vol. 151, no. 3, pp. 220-228, May 2011.
[27] Shen, H., Ackerman, A. L., Cody, V., Giodini, A., Hinson, E. R., Cresswell, P., Edelson, R. L., Saltzman, W. M., & Hanlon, D. J., “Enhanced and prolonged cross-presentation following endosomal escape of exogenous antigens encapsulated in biodegradable nanoparticles,” Immunology, vol. 117, no. 1, pp. 78-88, 2006.
[28] Ritprajak, P., Kaewraemruaen, C., & Hirankarn, N., “Current Paradigms of Tolerogenic Dendritic Cells and Clinical Implications for Systemic Lupus Erythematosus,” Cells 2019, Vol. 8, Page 1291, vol. 8, no. 10, p. 1291, October 2019.
[29] Marín, E., Cuturi, M. C., & Moreau, A., “Tolerogenic dendritic cells in solid organ transplantation: Where do we stand?,” Front. Immunol., vol. 9, no. FEB, p. 274, February 2018.
[30] Domogalla, M. P., Rostan, P. V., Raker, V. K., & Steinbrink, K., “Tolerance through education: How tolerogenic dendritic cells shape immunity,” Front. Immunol., vol. 8, no. DEC, December 2017.
[31] Mandla, S., Davenport Huyer, L., Wang, Y., & Radisic, M., “Macrophage Polarization with Angiopoietin-1 Peptide QHREDGS,” ACS Biomater. Sci. Eng., vol. 5, no. 9, pp. 4542-4550 September 2019.
[32] De Smedt, T., Van Mechelen, M., De Becker, G., Urbain, J., Leo, O., & Moser, M., “Effect of interleukin-10 on dendritic cell maturation and function,” Eur. J. Immunol., vol. 27, no. 5, pp. 1229-1235 May 1997.
[33] Lepenies, B., Lee, J., & Sonkaria, S., “Targeting C-type lectin receptors with multivalent carbohydrate ligands,” Adv. Drug Deliv. Rev., vol. 65, no. 9, pp. 1271-1281 August 2013.
[34] Porkolab, V., Pifferi, C., Sutkeviciute, I., Ordanini, S., Taouai, M., Thépaut, M., Vivès, C., Benazza, M., Bernardi, A., Renaudet, O., et al., “Development of C-type lectin-oriented surfaces for high avidity glycoconjugates: towards mimicking multivalent interactions on the cell surface,” Org. Biomol. Chem., vol. 18, no. 25, pp. 4763-4772 July 2020.
[35] Masson, J. D., Thibaudon, M., Belec, L., & Crépeaux, G., “Calcium phosphate: a substitute for aluminum adjuvants?,” Expert Rev. Vaccines, vol. 16, no. 3, pp. 289-299, March 2017.
[36] O'Hagan, D. T. & Fox, C. B., “New generation adjuvants-From empiricism to rational design,” Vaccine, vol. 33, no. S2, pp. B14-B20, June 2015.
[37] Brindle, N. P. J., Saharinen, P., & Alitalo, K., “SIGNALLING AND FUNCTIONS OF ANGIOPOIETIN-1 IN VASCULAR PROTECTION,” Circ. Res., vol. 98, no. 8, p. 1014 April 2006.
[38] Fu, J., Lee, K., Chuang, P. Y., Liu, Z. H., & He, J. C., “Glomerular endothelial cell injury and cross talk in diabetic kidney disease,” Am. J. Physiol.—Ren. Physiol., vol. 308, no. 4, p. F287, February 2015.
[39] Ali, O. A., Huebsch, N., Cao, L., Dranoff, G., & Mooney, D. J., “Infection-mimicking materials to program dendritic cells in situ,” Nat. Mater., vol. 8, no. 2, pp. 151-158, 2009.
[40] Kobayashi, D., Endo, M., Ochi, H., Hojo, H., Miyasaka, M., & Hayasaka, H., “Regulation of CCR7-dependent cell migration through CCR7 homodimer formation,” Sci. Rep., vol. 7, no. 1, pp. 1-14, 2017.
[41] Verbeke, C. S. & Mooney, D. J., “Injectable, Pore-Forming Hydrogels for In Vivo Enrichment of Immature Dendritic Cells,” Adv. Healthc. Mater., vol. 4, no. 17, pp. 2677-2687 December 2015.
[42] Howard, G. P., Verma, G., Ke, X., Thayer, W. M., Hamerly, T., Baxter, V. K., Lee, J. E., Dinglasan, R. R., & Mao, H. Q., “Critical Size Limit of Biodegradable Nanoparticles for Enhanced Lymph Node Trafficking and Paracortex Penetration,” Nano Res., vol. 12, no. 4, p. 837, April 2019.
[43] Thurston, G., Rudge, J. S., Ioffe, E., Papadopoulos, N., Daly, C., Vuthoori, S., Daly, T., Wiegand, S. J., & Yancopoulos, G. D., “The anti-inflammatory actions of angiopoietin-1,” EXS, no. 94, pp. 233-245, 2005.
[44] Baffert, F., Le, T., Thurston, G., & McDonald, D. M., “Angiopoietin-1 decreases plasma leakage by reducing number and size of endothelial gaps in venules,” Am. J. Physiol.—Hear. Circ. Physiol., vol. 290, no. 1, January 2006.
[45] Seok, S. H., Heo, J. I., Hwang, J. H., Na, Y. R., Yun, J. H., Lee, E. H., Park, J. W., & Cho, C. H., “Angiopoietin-1 Elicits Pro-Inflammatory Responses in Monocytes and

1. A hydrogel comprising a nanoparticle, a microparticle, at least one peptide on the surface of the nanoparticle, and at least one zwitterionic motif on the surface of the microparticle, wherein each of the nanoparticle and the microparticle comprises a polysaccharide.

2. The hydrogel of claim 1, wherein the polysaccharide is dextran.

3. The hydrogel of claim 1, wherein the at least one peptide comprises insulin or GAD65 or the amino acid sequence GGGQHREDGS (SEQ ID NO: 1) or QHREDGS (SEQ ID NO: 2).

4. The hydrogel of claim 1, wherein a mean diameter of the microparticle is at least about 50 μm.

5. The hydrogel of claim 1, wherein a mean diameter of the nanoparticle is about 100 nm to about 300 nm.

6. The hydrogel of claim 1, wherein the zwitterionic motif is a metal organic framework (MOF).

7. The hydrogel of claim 6, wherein the metal organic framework is zeolitic imidazolate framework (ZIF) 8.

8. The hydrogel of claim 1, wherein the zwitterionic motif further comprises a cytokine.

9. The hydrogel of claim 8, wherein the cytokine is IL-10.

10. A method for inducing tolerance differentiation of a dendritic cell of a subject, the method comprising administering an effective amount of the hydrogel of claim 1 to the subject.

11. The method of claim 10, wherein the dendritic cell is in the skin of the subject.

12. The method of claim 10, wherein the administering is a subcutaneous administration.

13. The method of claim 10, wherein the subject has Type-1 diabetes.

14. The method of claim 10, further comprising releasing the at least one peptide from the surface of the nanoparticle in the subject over a time period of at least about 6 hours.

15. The method of claim 10, wherein the zwitterionic motif further comprises a cytokine.

16. The method of claim 15, further comprising releasing the cytokine from the zwitterionic motif in the subject over a time period of at least about 6 hours.
